(19) United States
(12) Patent Application Publication
(10) Pub. No.: US 2012/0231014 A1
(43) Pub. Date: Sep. 13, 2012

(54) Neural Regeneration

(75) Inventors: John G. Flanagan, Weston, MA (US); Alan Peter Tenney, New York, NY (US); Yingjie Shen, Brighton, MA (US); Jerry Silver, Bay Village, OH (US)

(73) Assignees: CASE WESTERN RESERVE UNIVERSITY, Cleveland, OH (US); PRESIDENT AND FELLOWS OF HARVARD COLLEGE, Cambridge, MA (US)

(21) Appl. No.: 13/391,210

(22) PCT Filed: Aug. 18, 2010
(86) PCT No.: PCT/US10/45859

§ 371 Date: May 30, 2012

(30) Foreign Application Priority Data
Aug. 18, 2009 (US) 61274520
Oct. 14, 2009 (US) 61251521

(51) Int. Cl. A61K 39/395; A61P 25/00; A61K 38/46; A61K 38/17; C12N 5/0793; C12N 5/071

(52) U.S. Cl. 424/158.1; 435/375; 424/94.6; 514/17.7; 424/172.1

(57) ABSTRACT

A method of promoting neural cell regeneration is carried out by contacting a neural cell with a compound that inhibits the binding of a chondroitin sulfate proteoglycan (CSPG) to a cellular (e.g., trans-membrane) PTPσ protein. The neural cell is associated with an injury or neurodegenerative condition.

### RELATED APPLICATIONS

This application claims the benefit of priority of U.S. Patent Application Ser. No. 61/274,520, filed 18 Aug. 2009, and Ser. No. 61/251,521, filed 14 Oct. 2009, the contents of which are incorporated herein by reference in their entirety.

### GOVERNMENTAL SUPPORT

This invention was made with Government support under grants HD29417, EY11559 and NS40043, awarded by the National Institutes of Health. The U.S. Government has certain rights in the invention.

### FIELD OF THE INVENTION

The invention relates to the treatment of neuronal injury.

### BACKGROUND OF THE INVENTION

Spinal cord injury and other central nervous system (CNS) injuries can cause permanent disability or loss of movement (paralysis) and sensation below the site of the injury. Recovery after CNS injury is minimal, leading to substantial current interest in potential strategies to overcome this challenge. A fundamental obstacle facing efforts to improve neuronal function after injury is the inability of the adult CNS to regenerate.

Two well-known classes of regeneration inhibitors are myelin-associated inhibitors (MAG, Nogo and OMGP); and inhibitors in scar tissue formed by glia at the injury site (e.g., chondroitin sulfate proteoglycan (CSPG)). CSPG is involved not only in traumatic injury, but also many other CNS diseases including neurodegeneration. Example receptors for myelin-associated inhibitors include Pir B and NgR.

CSPG present a barrier to axon regeneration, yet no specific receptor for the inhibitory effect of CSPG has been identified previously. More specifically, CSPG shows dramatic upregulation after neural injury, both within the extracellular matrix of scar tissue and in the perineuronal net within more distant targets of the severed axons. The inhibitory nature of CSPG is not only reflected in the formation of dystrophic axonal retraction bulbs that fail to regenerate through the lesion, but also in the limited ability for collateral sprouting of spared fibers. Although it has been known for nearly two decades that sulfated proteoglycans are major contributors to the repulsive nature of the glial scar, the precise inhibitory mechanism was poorly understood. Thus, there remains an urgent need for mechanisms that modulate CSPG function.

### SUMMARY OF THE INVENTION

The present invention provides for a transmembrane protein tyrosine phosphatase, protein tyrosine phosphatase sigma (PTPσ), that binds with high affinity to neural CSPGs. Thus, PTP is the first-identified receptor for the scar tissue-associated regeneration inhibitors. CSPG binding involves the CS chains and a specific site on the first immunoglobulin-like domain of PTPσ. In culture, PTPσ−/− neurons show reduced inhibition by CSPG. A PTPσ fusion protein probe can detect cognate ligands upregulated specifically at neural lesion sites. After spinal cord injury, PTPσ gene disruption enhancesd the ability of axons to penetrate regions containing CSPG. These results indicate that PTPσ can act as a receptor for CSPGs, and may provide new therapeutic approaches to neural regeneration.

Aspects of the present invention are based on the discovery that CSPG, an endogenous barrier to axon regeneration, binds to a transmembrane PTPσ in the body and as a result inhibits neural regeneration. Accordingly, this interaction is inhibited, reduced or blocked as a therapeutic approach to increase neural regeneration.

Also within the invention is a method of identifying a compound that reduces CSPG inhibition of neuronal outgrowth. Such a method is carried out, for example, by providing a polypeptide comprising first immunoglobulin-like domain of PTPσ and contacting the polypeptide with a candidate compound. Binding of the candidate compound to the polypeptide indicates that the candidate compound reduces binding of CSPG to PTPσ, thereby reducing CSPG inhibition of neuronal outgrowth.

In addition to the therapeutic methods described above, the invention includes a method of identifying a site or incident of neural injury by administering to a subject a detectably-labeled PTPσ or a fragment thereof. For example, the fragment comprises the first immunoglobulin-like domain of PTPσ. Localization of the PTPσ or fragment thereof at an anatomical location indicates that the location is a site of neural injury.

The compositions and methods described herein are useful to promote neuronal outgrowth and treat spinal cord injury and other forms of neuronal trauma and degeneration, and offer advantages to earlier approaches, because they address the body's endogenous barriers to neuronal outgrowth (e.g., regeneration).

One aspect of the present invention relates to a method of promoting outgrowth of a neuron, comprising contacting the neuron with an agent that inhibits the interaction of chondroitin sulfate proteoglycan (CSPG) to protein tyrosine phosphatase sigma (PTPσ). In one embodiment, the neuron is in vivo. In one embodiment, the neuron is in vitro.

One aspect of the present invention relates to a method of promoting neural regeneration in the CNS of a subject in need thereof, comprising contacting a neuron of the subject with an agent that inhibits the interaction of chondroitin sulfate proteoglycan (CSPG) to PTPσ. The agent may inhibit binding of CSPG to PTPσ. The neuron may be located at a site of injured or diseased tissue, e.g., at a site of spinal cord injury. The agent may bind to the first immunoglobulin-like domain of PTPσ. The agent may comprise a soluble PTPσ polypeptide or fragment thereof. The agent may comprise a soluble PTPσ ectodomain or fragment thereof. In one embodiment, the agent comprises a molecule that binds to the CSPG binding site of PTPσ, or that binds to the PTPσ binding site of CSPG. In one embodiment, the agent is a PTPσ-specific antibody or fragment thereof, the epitope binding specificity of which comprises the first immunoglobulin-like domain of PTPσ. The method may further comprise contacting the cell with a second agent, wherein the second agent inhibits a myelin inhibitor of neural regeneration.

Another aspect of the present invention relates to a method of identifying a compound that reduces CSPG inhibition of neural regeneration, comprising providing a polypeptide comprising a PTPσ polypeptide or fragment thereof that binds to CSPG, contacting the polypeptide with a candidate compound, wherein binding of the candidate compound to the polypeptide indicates that the candidate compound reduces binding of CSPG to PTPσ, thereby reducing CSPG inhibition of neural regeneration. In one embodiment, the fragment comprises the first immunoglobulin-like domain of PTPσ.

Another aspect of the present invention relates to a method of identifying a site of neural injury by administering to a subject a detectably-labeled PTPσ or a fragment thereof which binds to CSPG, wherein localization of the PTPσ or fragment thereof at an anatomical location indicates that the location is a site of neural injury. The fragment may comprise the first immunoglobulin-like domain of PTPσ.

Another aspect of the present invention relates to a method of inhibiting the interaction of chondroitin sulfate proteoglycan (CSPG) to-PTPσ in a cell, comprising contacting the cell with an agent that inhibits CSPG binding to PTPσ. The cell may be a neuron, e.g., a central nervous system neuron or a peripheral nervous system neuron. The cell may be a non-neuronal cell that provides support to neural cells. The agent may be isolated/purified CSPG or a fragment thereof. The fragment of CSPG is chondroitin sulfate or a fragment thereof. The agent may be isolated/purified antibody which specifically binds an epitope on CSPG. The epitope may be on chondroitin sulfate. The agent may be an isolated/purified antibody or fragment thereof which specifically binds an epitope on immunoglobulin-like domain of PTPσ. The epitope may be on the first immunoglobulin-like domain of PTPσ. The agent may be an isolated/purified soluble PTPσ polypeptide. The agent may be a purified/isolated soluble PTPσ ectodomain. The agent may be a small molecule.

### DESCRIPTION OF THE DRAWINGS

FIG. 1A-1G shows the binding of PTPσ ectodomain to CSPG. (1A) Domain structure of PTPσ and the CSPG neurocan. ΔLys indicates the site in PTPσ where a cluster of lysines were mutated. (1B-1E) Purified recombinant PTPσ-Fc fusion protein was immobilized, and treated with neurocan-AP fusion, or AP tag control, followed by quantitation of bound AP activity. (1B) Neurocan-AP bound above control. (1C) The PTPσ ΔLys mutation reduced binding to background levels. Pretreatment of neurocan-AP with chondroitinase ABC (ChABC) reduced binding. (1D) Binding between PTPσ-Fc and neurocan-AP was saturable. (1E) Scatchard analysis produced a linear plot indicating a single binding affinity with K_{D}=11 nM. (1F) PTPσ-AP bound to C8-D1A astrocyte cultures above AP control. Binding was reduced by anti-CS antibody, but not IgM control. (1G) Chondroitinase ABC pretreatment of astrocyte cultures reduced PTPσ-AP binding. Western blot showed bands with expected sizes for neurocan proteolytic fragments at approximately 150 kD and 100 kD (asterisks). ***p<0.001; **p<0.01. See Shen et al., PTPσ is a receptor for chondroitin sulfate proteoglycan, an inhibitor of neural regeneration, 326 Science 592 (2009) for color versions of figures.

FIG. 2A-C shows the effect of PTPσ deficiency on the response of sensory neurons to CSPG. (2A) Quantitation of neurite outgrowth. PTPσ^{-/-} neurons showed significantly less inhibition by CSPG than wild type neurons. A significant difference was no longer seen when chondroitinase ABC was added along with the CSPG. (2B, 2C) Effect of CSPG or MAG, expressed as percent inhibition of outgrowth. PTPσ deficiency reduced the inhibitory action of CSPG, but had no significant effect on the inhibitory action of MAG. n=5 mice for each genotype. *p<0.05, **p<0.01. Scale bars, 100 μm.

FIG. 3A-3B demonstrates that adult PTPσ^{-/-} sensory neurons show reduced sensitivity to inhibition by a proteoglycan gradient and can extend further in a spinal cord lesion. (3A) Quantitation of average number of axons per spot growing up the gradient and crossing the outer rim. Wild type n=48, PTPσ^{-/-} n=40 spots; ***p<0.001. (3B) Quantitation of distance from lesion center. Wild type n=35, PTPσ^{-/-} n=30 mice. ***p<0.002.

FIG. 4A-4D shows binding of aggrecan or CS to the PTPσ ectodomain. Aggrecan or CS were biotinylated, and were detected with streptavidin-AP after binding to substrate-anchored PTPσ-Fc. (4A) Aggrecan bound to PTPσ-Fc at levels higher than to Fc control. The PTPσ-ΔLys mutant did not show binding significantly above control level. (4B) Aggrecan binding to PTPσ-Fc was saturable. A hyperbolic curve fit produced a K_{D} of 19 nM. (4C) Isolated CS bound to PTPσ-Fc at levels higher than to Fc control. The PTPσ ΔLys mutant did not show binding significantly above control level. (4D) CS binding to PTPσ-Fc was saturable. Because the CS preparation is heterogeneous in molecular weight, its molarity and therefore the K_{D} cannot be calculated precisely in this experimental format. These data fit well to a hyperbolic binding curve (R²=0.95), however, and based on a typical 10-50 kDa size range of CS preparations, the K_{D} is likely to be in the nanomolar range. Considering that binding energy is related to the logarithm of K_{D} (G=−RT.lnK), these data suggest it is likely that most or all of the binding energy for the interaction of PTPσ with CSPGs comes from binding to the CS chains. It is possible that PTPσ could also interact with the core protein of neurocan, aggrecan or other CSPGs. Error bars, SEM. ***p<0.001, Student's t test.

FIG. 5 demonstrates that PTPσ^{-/-} neurons showed less inhibition by neurocan than wild type neurons. Bar graph shows the effect of neurocan, expressed as percent inhibition of outgrowth. Error bars, SEM. n=3 mice for each genotype. ***p<0.001, Student's t test.

FIG. 6 shows PTPσ^{-/-} and wild type neurons did not show significantly different outgrowth with NGF stimulation. DRG neurons from postnatal day 8-12 wild type or PTPσ^{-/-} mice were dissociated and plated on slides coated with poly-D-lysine. 30 minutes after seeding, cells were treated with or without 50 ng/ml NGF, and were cultured for an additional 24 hours before immunostaining. Total length of GAP-43 positive neurites was quantified with Metamorph software. Error bars, SEM. n=3 mice for each genotype. Without NGF, p=0.67; with NGF, p=0.99; Student's t test.

FIG. 7 shows adult PTPσ/sensory neurons can extend further into a spinal cord lesion: additional quantitation method. Animals received a dorsal column crush injury, followed by axon tracing with DexTR, as described in FIG. 3. The bar graph shows the quantitation of the distance of wild type or PTPσ^{-/-} axon fiber front from the lesion center. Wild type n=80, PTPσ^{-/-} n=53 sections; error bars, SEM; ***p<0.0001, Student's t test.

### DETAILED DESCRIPTION

The present invention is not limited to the particular methodology, protocols, and reagents, etc., described herein and as such may vary. The terminology used herein is for the purpose of describing particular embodiments only, and is not intended to limit the scope of the present invention, which is defined solely by the claims. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

Unless otherwise defined, scientific and technical terms used in connection with the antibodies described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

As used herein, the term axonal “growth” or “outgrowth” (also referred to herein as “neuronal outgrowth”) includes the process by which axons or dendrites extend from a neuron. The outgrowth can result in a new neuritic projection or in the extension of a previously existing cellular process. Axonal outgrowth may include linear extension of an axonal process by five cell-diameters or more. Neuronal growth processes, including neuritogenesis, can be evidenced by GAP-43 expression detected by methods such as immunostaining. “Stimulating axonal growth” means promoting axonal outgrowth.

“Central nervous system (CNS) neurons” include the neurons of the brain, the cranial nerves and the spinal cord. The term CNS neuron is not intended to include support-cells or protection-cells such as astrocytes, oligodentrocytes, microglia, ependyma and the like, nor is it intended to include peripheral nervous system (e.g., somatic, autonomic, sympathetic or parasympathetic nervous system) neurons. Although, in some embodiments, such cells are also, contacted with the agents described herein.

“Peripheral nervous system (PNS) neurons” includes the neurons which reside or extend outside of the CNS. PNS is intended to include the neurons commonly understood as categorized in the peripheral nervous system, including sensory neurons and motor neurons. The term PNS neuron is not intended to include support or protection cells such as Schwann cells, satellite glia, enteric glia, and the like, nor is it intended to include CNS nervous system neurons, although, in some embodiments, such cells are also contacted with the agents described herein.

The term “patient” or “subject” or “animal” or “host” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The agents, compounds, compositions, antibodies, etc. used in the methods of the invention are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

An “effective amount” of an agent is an amount sufficient to achieve a desired therapeutic or pharmacological effect, such as an amount sufficient to inhibit the binding of CSPG to PTPσ, or an amount that is capable of activating the growth of neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

A “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.”

“Administering” to a patient includes dispensing, delivering or applying an active compound in a pharmaceutical formulation to a subject by any suitable route for delivery of the active compound to the desired location in the subject (e.g, to thereby contact a desired cell such as a desired neuron), including administration into the cerebrospinal fluid or across the blood-brain barrier, delivery by either the parenteral or oral route, intramuscular injection, subcutaneous or intraderrnal injection, intravenous injection, buccal administration, transdermal delivery and administration by the rectal, colonic, vaginal, intranasal or respiratory tract route. The agents may, for example, be administered to a comatose, anesthetized or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

The term “inhibit”, refers to inhibiting an interaction or inhibiting binding (e.g., to result in the inhibition of the binding of CSPG with PTPσ), is used to refer to a reduction in an activity (e.g., binding or promotion/inhibition of signal transduction) by a substantial amount, in the presence of an agent. Such an amount is detectable, reproducible and statistically or near statistically significant, as compared to the interaction in the absence of the agent. For example, a reduction in the interaction (e.g., binding) such that the observed/detected interaction in the presence of the agent is 90% of the interaction as observed/detected in the absence of the agent, using a given detection method. This would be a 10% reduction. In one embodiment, a 100% reduction of the interaction is achieved. If the interaction were binding, then no detectable binding would occur in the presence of the inhibiting agent. A smaller reduction in the interaction would also be considered useful (e.g., 90%, 80%, 70%, 60%, 50%, 40%, 30%, 20%, or 10%, inclusive). A 10% reduction, or less, (e.g., 9%, 8%, 7%, 6%, 5% or less, inclusive) is also expected. Detection of the interaction can be by any number of methods or assays. For example, functional assays which detect neuronal outgrowth or PTPσ signaling can be used. More direct assays can be used as well, such as direct binding assays (co-immunoprecipitation).

As used herein, the term “Neurological disorder” includes a disease, disorder, or condition which directly or indirectly affects the normal functioning or anatomy of a subject's nervous system. The term “stroke” is art-recognized and includes sudden diminution or loss of consciousness, sensation and voluntary motion caused by rupture or obstruction (for example, by a blood clot) of an artery of the brain. “Traumatic brain injury” is art-recognized and includes the condition in which a traumatic blow to the head causes damage to the brain or connecting spinal cord, with or without penetrating the skull. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

The term “antibody”, includes human and animal mAbs, and preparations of polyclonal antibodies, synthetic antibodies, including recombinant antibodies (antisera), chimeric antibodies, including humanized antibodies, anti-idiotopic antibodies and derivatives thereof. A portion or fragment of an antibody refers to a region of an antibody that retains at least part of its ability (binding specificity and affinity) to bind to a specified epitope. The term “epitope” or “antigenic determinant” refers to a site on an antigen to which antibody paratope binds. Epitopes formed from contiguous amino acids are typically retained on exposure to denaturing solvents whereas epitopes formed by tertiary folding are typically lost on treatment with denaturing solvents. An epitope typically includes at least 3, at least 5, or 8 to 10, or about 13 to 15 amino acids in a unique spatial conformation. Methods of determining spatial conformation of epitopes include, for example, x-ray crystallography and 2-dimensional nuclear magnetic resonance. See, e.g., 66 EPITOPE MAPPING PROTOCOLS IN METS. IN MOLECULAR BIO. (Morris, ed., 1996); Burke et al., 170 J. Inf. Dis. 1110-19 (1994); Tigges et al., 156 J. Immunol. 3901-10).

The terms “portion”, “fragment”, “variant”, “derivative” and “analog”, when referring to a polypeptide of the present invention include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

The terms “peptide” or “polypeptide”, as used herein, refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, N.Y., 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green & Wuts, PROTECTING GROUPS IN ORGANIC SYNTHESIS (John Wiley & Sons, 1991). The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can antagonize CSPG-PTPσ interaction. Such peptide antagonists can then be isolated by suitable means.

The term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of normatural amino acids).

A “small molecule”, as used herein, refers to a low molecular weight organic compound that is, by definition, not a polymer. In one embodiment, the small molecule is a molecule that also binds with high affinity to a biopolymer such as protein, nucleic acid or polysaccharide, and in addition alters the activity or function of the biopolymer. The upper molecular weight limit for a small molecule is approximately 800 D, which allows for the possibility rapid diffuse across cell membranes so that they can reach intracellular sites of action.

“Contacting neurons” refers to any mode of agent delivery or “administration,” either to cells or to whole organisms, in which the agent is capable of exhibiting its pharmacological effect in neurons. “Contacting neurons” includes both in vivo and in vitro methods of bringing an agent of the invention into proximity with a neuron. Suitable modes of administration can be determined by those skilled in the art and such modes of administration may vary between agents. For example, when axonal growth of neurons is stimulated ex vivo, agents can be administered, for example, by transfection, lipofection, electroporation, viral vector infection, or by addition to growth medium.

Recovery after CNS injury is minimal, leading to substantial current interest in potential strategies to overcome this challenge. Case & Tessier-Lavigne, 15 Curr. Bio. R749 (2005); Domeniconi et al., 233 J. Neurol. Sci. 43 (2005); Liu et al., 361 Phios. Trans. R Soc. Lond. B Bio. Sci. 1593 (2006); Lu et al., 209 Exp. Neurol. 313 (2008); Yiu & He, 7 Nat. Rev. Neurosci. 617 (2006). CSPGs show dramatic upregulation after neural injury, within the extracellular matrix of scar tissue and in the perineuronal net within more distant targets of the severed axons. Silver & Miller, 5 Nat. Rev. Neurosci. 146 (2004); Rhodes & Fawcett, 204 J. Anat. 33 (2004). The inhibitory nature of CSPGs is not only reflected in formation of dystrophic axonal retraction bulbs that fail to regenerate through the lesion (but also in the limited ability for collateral sprouting of spared fibers. Tom et al., 24 J. Neurosci. 6531 (2004); Massey et al., 26 J. Neurosci. 4406 (2006). This inhibition can be relieved by chondroitinase ABC digestion of the CS sidechains, which can promote regeneration/sprouting and restore lost function. Bradbury et al., 416 Nature 636 (2002); Cafferty et al., 28 J. Neurosci. 11998 (2008); Houle et al., 26 J. Neurosci. 7405 (2006); Pizzorusso et al., 103 PNAS 8517 (2006); Tester & Howland, 209 Exp. Neurol. 483 (2008). Although it has been known for nearly two decades that sulfated proteoglycans are major contributors to the repulsive nature of the glial scar (Snow et al., 109 Exp. Neurol. 111 (1990)), the precise inhibitory mechanism was poorly understood. Because the identification of specific neuronal receptors for CSPGs had been lacking, relatively non-specific mechanisms brought about by arrays of negatively charged sulfate (Gilbert et al., 29 Mol. Cell. Neurosci. 545 (2005)), or occlusion of substrate adhesion molecules (McKeon et al., 136 Exp. Neurol. 32 (1995)), were suggested.

Aspects of the invention relate to the promotion of neuronal outgrowth by contacting a neuron with an agent that inhibits the interaction of chondroitin sulfacte proteoglycan (CSPG) with Protein Tyrosine Phosphatase sigma (PTPσ). This interaction inhibits neuronal outgrowth (e.g., the neuronal outgrowth involved in neural regeneration). Inhibition of the interaction (e.g., inhibition of binding of CSPG to PTPσ) prevents the inhibition of neuronal outgrowth by CSPG, and thereby promotes a regenerative effect. Without being bound by theory, it is thought that removal of endogenous inhibition of neuronal outgrowth, allows endogenous activation of neuronal outgrowth to proceed. The neuronal outgrowth which ensues can be neuronal regeneration, (e.g., of an injured neuron or a nerve that has undergone neurodegeneration) or can be outgrowth of a healthy neuron to produce sprouting or plasticicity of compensatory connections (e.g., to compensate for an injured neuron).

One aspect of the invention relates to a method for promoting neural regeneration by contacting a neuron with an agent that inhibits the interaction of chondroitin sulfacte proteoglycan (CSPG) with Protein Tyrosine Phosphatase sigma (PTPσ). The interaction of CSPG with PTPσ which is to be disrupted inhibits neuronal outgrowth.

Such an effect of the disruption of the interaction (regenerative or compensatory growth) is evidenced, for example, by axonal outgrowth of a neuron, or by increased responsiveness to an agent that promotes neuronal outgrowth of a neuron. As such, one aspect of the invention relates to a method for promoting axonal outgrowth of a neuron. The method comprises contacting the neuron with an agent inhibits the interaction of CSPG with PTPσ. In one embodiment, the neuron is also contacted with an agent that actively promotes neuronal outgrowth of a neuron.

The identification of a specific site on PTPσ that binds CSPG represents a site of intervention for drugs to treat spinal cord injury and other neuronal injuries and disorders. Blocking approaches include, for example using soluble receptor ectodomains. Such approaches are optionally combined with blockade of other regeneration inhibitors. These therapeutic approaches are relevant to many other forms of neural injury as well as neurodegeneration that involve reactive astrogliosis. These therapeutic interventions enhance regeneration or plasticity after nervous system injury. As such, another aspect of the invention relates to treatment of a neurological disorder in a subject in need thereof, comprising administering to the subject a pharmaceutical composition comprising an effective amount of an agent that inhibits the interaction of CSPG with PTPσ. Administration is to thereby contact one or more cells of the subject to thereby promote neuronal outgrowth, to thereby treat the neurological disorder. The neurological disorder may result from injury (e.g., traumatic injury) or from neurodegenerative disease.

It has also been discovered that an agent that binds CSPG (a binding agent of CSPG) can detect lesion sites in the adult CNS. This finding not only sheds light on the biological role of CSPG, but also provides an injury biomarker, and thus a tool for research or diagnosis. As such, another aspect of the present invention relates to a method of identifying a site of neural injury in a subject. The method comprises administering to the subject a binding agent of CSPG and detecting sites of binding of the binding agent. Sites where binding is significantly higher, as compared to binding in an appropriate control, are indicated as having increased amounts of CSPG. These sites correspond to a site of neuronal injury. As such, detection and localization of increased binding of the binding agent at an anatomical location would thereby indicate that the location is a site of neuronal injury. In one embodiment, the binding agent is detectably labeled. In one embodiment, the binding agent is PTP or a fragment thereof which binds to CSPG. In one embodiment, the fragment comprises the first immunoglobulin-like domain of PTPσ. In one embodiment, the binding agent is a PTP fusion protein.

### Agents

The term “agent that inhibits the interation of CSPG to PTPσ” refers to inhibition of the interaction which leads to the inhibition of neuronal outgrowth. One such method of interfering with this interaction is by preventing binding of CSPG to PTPσ. As such, one such agent is an inhibitor of binding of CSPG to PTPσ. Agents that inhibit the interaction of CSPG to PTPσ (inhibiting neuronal outgrowth) include protein-based therapeutics such as proteins or polypeptides as well as small molecules.

One such agent is isolated CSPG or a portion or fragement thereof that specifically binds PTPσ. For example, CS has been shown to specifically bind PTPσ. In one embodiment, the fragment is CS. In another embodiment, the fragment is a smaller fragment of CS which retains binding and/or interaction with PTPσ. Such CSPG fragments can be generated, for example, by fragmentation of purified CSPG. Fragmentation can be achieved, for example by digestion (e.g., with chondroitinase). Following digestion, the desired fragments can be further purified from the digestion mixture. Alternatively, fragments can be isolated and purified from their natural state (e.g., cell culture). The CSPG or fragment thereof may be further modified, as described herein. In one embodiment, the CSPG or portion or fragment thereof is mammalian (e.g., human).

Another such agent is isolated PTPσ or a polypeptide fragment thereof that specifically binds CSPG. In one embodiment, the fragment comprises the PTPσ ectodomain or a portion or fragment thereof. In one embodiment, the fragment comprises one or more of the immunoglobulin-like domains of PTPσ. In one embodiment, the fragment comprises the first immunoglobulin-like domain of PTPσ. The PTPσ or fragment thereof may be in the form of a fusion protein. The fragment may further include moieties to increase solubility, purification, and/or delivery. One such way to modify a fragment is to synthesize it as a fusion protein. One such fusion protein is an Fc fragment fusion protein. Such Fc fragments, and generation of fusion proteins, are known in the art (see, e.g., U.S. Pat. No. 7,504,482). In one embodiment, the agent comprises a soluble PTPσ polypeptide such as a soluble purified PTPσ ectodomain or a soluble purified first immunoglobulin-like domain of a cell-bound PTPσ. In one embodiment, the PTPσ or fragment thereof is mammalian (e.g., mouse, human).

Another such agent is a peptidomimetic that inhibits the interaction of CSPG to PTPσ. One such peptidomimetic could be a peptidomimetic of a fragment of PTPσ that inhibits binding of CSPG to PTPσ. Peptidomimetics can be synthesized and assembled into libraries comprising a few to many discrete molecular species. Such libraries can be prepared using well known methods of combinatorial chemistry, and can be screened as described herein to determine if the library comprises one or more peptidomimetics which antagonize the CSPG-PTPσ interaction. Such peptidomimetic antagonists can then be isolated by suitable methods.

For example, polysaccharides can be prepared that have the same functional groups as peptides which can antagonize the interaction of CSPG to PTPσ. Peptidomimetics can be designed, for example, by establishing the three dimensional structure of a peptide agent in the environment in which it is bound or will bind to the target (e.g., CSPG or PTPσ). The peptidomimetic comprises at least two components, the binding moiety or moieties and the backbone or supporting structure.

The binding moieties are the chemical atoms or groups that react or form a complex (e.g., through hydrophobic or ionic interactions) with the target (e.g., PTPσ first Ig-like domain), for example, with the amino acid(s) at or near the ligand binding site. For example, the binding moieties in a peptidomimetic can be the same as those in a peptide antagonist of CSPG-PTPσ binding. The binding moieties can be an atom or chemical group which reacts with the receptor in the same or similar manner as the binding moiety in a peptide antagonist of CSPG-PTPσ binding. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the 3-dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more-CONH-groups for a-NHCO-group). In yet another embodiment, the peptide backbone can be substituted with a polysilane backbone.

These compounds can be manufactured by known methods. For example, a polyester peptidomimetic can be prepared by substituting a hydroxyl group for the corresponding a-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Another such agent is an antibody or fragment thereof that binds to an epitope on CSPG to thereby inhibit the interaction of CSPG with PTPσ (e.g., inhibit the binding). In one embodiment, the epitope is on CS. Another such agent is an antibody or fragment thereof that binds to an epitope on PTPσ to thereby inhibit the interaction of CSPG with PTPσ (e.g., inhibit the binding). In one embodiment, the epitope is on the ectodomain. In one embodiment, the epitope is on one of the Ig-like domains. In one embodiment, the epitope is on the first Ig-like domain.

Another such agent is a small molecule. The small molecule may bind to CSPG or to PTPσ. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof.

An effective amount of the agents is used in the various embodiments of the invention. An effect amount is an amount that results, at least partially or substantially, in the inhibition of the interaction of CSPG with PTPσ.

### Other Agents

The methods described herein can further include administration or contacting a cell (e.g., a neuron) with a second agent that blocks other regeneration inhibitors, e.g., a compound that inhibit myelin derived blockage of neural generation. Known inhibitors of neuronal outgrowth (e.g., of regeneration at a CNS injury site) are myelin-derived inhibitors (e.g., Nogo-A, MAG, OMgp, Ehprin B3, Sema 4D and Sema 5A), astrocyte derived inhibitors (e.g., CSPG, KSPG, Ephrin B2 and Slit), fibroblast derived inhibitors (e.g., Sema 3A). The second agent may be an antagonist to any of these inhibitors. In one embodiment, the cell is further contacted with one or more such second agents. In one embodiment, the agent inhibits a myelin inhibitor of neural regeneration (e.g., myelin-associated glycoprotein (MAG), Nogo, oligodendrocyte myelin glycoprotein (OMgp)). Inhibitors of MAG are disclosed in U.S. Pat. No. 5,932,542. Inhibitors of Nogo are disclosed in U.S. Patent Application Pub No. 2009/0215691. Inhibitors of OMgp are disclosed in U.S. Patent Application Pub. No. 2008/0188411. The cell can be contacted with this second agent before, after, and/or concurrently with the agent that inhibits the interaction of CSPG with PTPσ.

The methods described herein can further include administration or contacting a cell (e.g., a neuron) with one or more agents that stimulate neuronal outgrowth. Such agents promote the intrinsic growth capability of a neuron. The cell can be contacted with this second agent before, after, and/or concurrently with the agent that inhibits the interaction of CSPG with PTPσ and/or before, after, and/or concurrently with the second agent that blocks other regeneration inhibitors. Such agents which promote neuronal outgrowth are known in the art. Such agents include, without limitation, agents that activate the growth pathway of neurons (e.g., CNS) are agents that are capable of producing a neurosalutary effect. As used herein, a “neurosalutary effect” means a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The phrase “producing a neurosalutary effect” includes producing or effecting such a response or improvement in function or resilience within a component of the nervous system. For example, examples of producing a neurosalutary effect would include stimulating axonal outgrowth after injury to a neuron; rendering a neuron resistant to apoptosis; rendering a neuron resistant to a toxic compound such as β-amyloid, ammonia, or other neurotoxins; reversing age-related neuronal atrophy or loss of function; or reversing age-related loss of cholinergic innervation.

Any agent that activates the growth pathway of neurons (e.g., CNS) is suitable for use in the methods of the present invention in combination with an agent inhibits the interaction of CSPG with PTPσ. Some agents include but are not limited to neurotrophic factors such as inosine, mannose, gulose, or glucose-6-phosphate, as described in Li et al., 23 J. Neurosci. 7830 (2003); Chen et al., 99 PNAS 1931 (2002); and Benowitz et al., 273 J. Biol. Chem. 29626 (1998). TGF-β, and oncomodulin as described in Yin et al., 23 J. Neurosci. 2284 (2003), are also agents. In addition, polypeptide growth factors such as BDNF, NGF, NT-3, CNTF, LIF, and GDNF can be used. In one embodiment the methods of the present invention which comprise an agent that stimulates neuronal outgrowth further comprise contacting neurons (e.g., CNS) with a cAMP modulator that increases the concentration of intracellular cAMP (e.g., cAMP), and/or polyamines (Cai et al., 35 Neuron 711 (2002)). For example, the ability of mature rat retinal ganglionic cells to respond to mannose requires elevated cAMP (Li et. al., 2003).

The agents described herein may further be modified (e.g., chemically modified). Such modification may be designed to facilitate manipulation or purification of the molecule, to increase solubility of the molecule, to facilitate administration, targeting to the desired location, to increase or decrease half life. A number of such modifications are known in the art and can be applied by the skilled practitioner.

### Binding Agents of CSPG

Binding agents of CSPG are used to detect CSPG in an organism. Such agents are useful to thereby detect the presence of CSPG as an indicator of neuronal damage. Such binding agents include agents which bind to CSPG to inhibit the binding with PTPσ, described herein. The binding agent need not inhibit the interaction of CSPG with PTPσ, however, as long as it specifically binds CSPG sufficiently to be useful in the quantitative localization of CSPG. In one embodiment, the binding agent is an antibody, or fragment thereof, which specifically binds an epitope on CSPG. In another embodiment, the antibody or fragment thereof specifically recognizes an epitope on CS.

In one embodiment, the binding agent is detectably labeled. In one embodiment, the detectable label is a label useful in medical imaging, to thereby allow detection of neuronal damage in a live subject. Detection of the damage will greatly facilitate treatment. For example, detection of the site of damage can indicate a site for targeting of the therapeutic agents described herein. Such imaging and labels are known in the art. U.S. Patent Application Pub. No. 2008/0075661 provides information regarding diagnostic medical imaging and labels useful in the detection of the CSPG via the binding agent of CSPG described herein.

### Targeted Cells

The agents and therapeutic pharmaceutical compositions described herein may be delivered to neurons of the CNS and/or the PNS. Such neurons may be injured or diseased. Such neurons may alternatively be healthy, uninjured neurons. Such neurons may be located at the site of injury, or at a site incident to the injury. The neurons to be targeted for therapeutic administration, delivery/contact of the agents and compositions described herein will be neurons from which neuronal outgrowth is thought to prove beneficial to the subject. Such determination is within the ability of the skilled practitioner through no more than routine experimentation.

The agents and therapeutic pharmaceutical compositions described herein may also be delivered to non-neuronal cells of the CNS and/or the PNS, such as to non-neuronal cells that provide support to neural cells. Such cells include, without limitation, glial cells (e.g., astrocytes, oligodendrocytes, ependymal cells, radial glia in the CNS; and Schwann cells, satellite glial cells, enteric glail cells n the PNS).

### Antibodies

Antibodies useful in the present invention bind to the specified epitopes. Single-chain antibodies, chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. No. 4,816,567; U.S. Pat. No. 4,816,397; U.S. Pat. No. 5,225,539; EP 0,125,023 B1; EP 0,120,694 B1; WO 86/01533; EP 0,194,276 B1; EP 0,239,400 B1; EP 0451216 B1; EP 0519596 A1. See also, Newman et al., 10 BioTech. 1455 (1992), regarding primatized antibody; and U.S. Pat. No. 4,946,778 and Bird et al., 242 Science 423 (1988) regarding single-chain antibodies.

Humanized antibodies can be produced using synthetic or recombinant DNA technology using standard methods or other suitable techniques well-known in the art. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman et al., 17 Nucl. Acids Res. 5404 (1989); Sato et al., 53 Cancer Res. 851 (1993); Daugherty et al., 19 Nucleic Acids Res. 2471 (1991); Lewis & Crowe, 101 Gene 297 (1991)). Using these or other suitable methods, variants can also be produced readily. In one embodiment, cloned variable regions can be mutated, and sequences encoding variants with the desired specificity can be selected (e.g., from a phage library; see e.g., U.S. Pat. No. 5,514,548; WO 93/06213.

Antibodies that are specific for mammalian (e.g., human) PTPσ can be raised against an appropriate immunogen, such as isolated and/or recombinant human PTPσ or portions thereof (including synthetic molecules, such as synthetic peptides).

Preparation of immunizing antigen, and polyclonal and monoclonal antibody production can be performed using any suitable technique. For example, monoclonal antibodies directed against binding cell surface epitopes can be readily produced by one skilled in the art. The general methodology for making monoclonal antibodies by hybridomas is well known. Other suitable methods of producing or isolating antibodies of the requisite specificity can be used, including, for example, methods which select recombinant antibody from a library (e.g., a phage display library). Transgenic animals capable of producing a repertoire of human antibodies (e.g., XENOMOUSE™ mice from Abgenix, Fremont, Calif.) can be produced using suitable methods (see, e.g., WO 98/24893; U.S. Pat. No. 5,939,598; Jakobovits et al., 90 PNAS 2551 (1993); Jakobovits et al., 362 Nature 255 (1993)). Additional methods for production of transgenic animals capable of producing a repertoire of human antibodies have been described (see e.g., U.S. Pat. No. 5,545,806; No. 5,545,807; WO 97/13852).

### Administration of the Pharmaceutically Acceptable Formulations to a Patient

In the methods of treatment disclosed herein, a therapeutically effective amount is administered to the subject. In one embodiment, the active compound formulation described herein is administered to the subject in the period from the time of, for example, an injury to the nervous system up to about 100 hours after the injury has occurred, for example within 24, 12, or 6 hours from the time of injury.

The compositions are administered such as the agents come into contact with a subject's nervous system. In one embodiment, the administration is specific for one or more specific locations within the subject's nervous system. The preferred mode of administration can vary depending upon the particular agent chosen and the particular target.

When the agents are delivered to a subject, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated. Agents can also be delivered using viral vectors, which are well known to those skilled in the art.

Both local and systemic administration are contemplated by the invention. Desirable features of local administration include achieving effective local concentrations of the active compound as well as avoiding adverse side effects from systemic administration of the active compound. In one embodiment, the active agents are administered by introduction into the cerebrospinal fluid of the subject. In certain aspects of the invention, the active compound is introduced into a cerebral ventricle, the lumbar area, or the cistema magna. In another aspect, the active compound is introduced locally, such as into the site of nerve or cord injury, into a site of pain or neural degeneration, or intraocularly to contact neuroretinal cells.

The pharmaceutically acceptable formulations can be suspended in aqueous vehicles and introduced through conventional hypodermic needles or using infusion pumps.

In another embodiment of the invention, the active compound formulation is administered into a subject intrathecally. As used herein, the term “intrathecal administration” is intended to include delivering an active compound formulation directly into the cerebrospinal fluid of a subject, by techniques including lateral cerebroventricular injection through a burrhole or cistemal or lumbar puncture or the like (described in Lazorthes et al., 1991, and Ommaya, 1984, the contents of which are incorporated herein by reference). The term “lumbar region” is intended to include the area between the third and fourth lumbar (lower back) vertebrae. The term “cistema magna” is intended to include the area where the skull ends and the spinal cord begins at the back of the head. The ten-n “cerebral ventricle” is intended to include the cavities in the brain that are continuous with the central canal of the spinal cord. Administration of an active compound to any of the above mentioned sites can be achieved by direct injection of the active compound formulation or by the use of infusion pumps. Implantable or external pumps and catheter may be used.

For injection, the active compound formulation of the invention can be formulated in liquid solutions, typically in physiologically compatible buffers such as Hank's solution or Ringer's solution. In addition, the active compound formulation may be formulated in solid form and re-dissolved or suspended immediately prior to use. Lyophilized forms are also included. The injection can be, for example, in the form of a bolus injection or continuous infusion (such as using infusion pumps) of the active compound formulation.

In one embodiment of the invention, the active compound formulation is administered by lateral cerebroventricular injection into the brain of a subject, usually within 100 hours of when an injury (resulting in a condition characterized by aberrant axonal outgrowth of central nervous system neurons) occurs (such as within 6, 12, 24 or 100 hours, inclusive, from the time of the injury). The injection can be made, for example, through a burr hole made in the subject's skull. In another embodiment, the formulation is administered through a surgically inserted shunt into the cerebral ventricle of a subject, usually within 100 hours of when an injury occurs (e.g., within 6, 12 or 24 hours, inclusive, from the time of the injury). For example, the injection can be made into the lateral ventricles, which are larger, even though injection into the third and fourth smaller ventricles can also be made. In yet another embodiment, the active compound formulation is administered by injection into the cistema magna, or lumbar area of a subject, within 100 hours of when an injury occurs (such as within 6, 12, or 24 hours, inclusive, from the time of the injury).

An additional means of administration to intracranial tissue involves application of compounds of the invention to the olfactory epithelium, with subsequent transmission to the olfactory bulb and transport to more proximal portions of the brain. Such administration can be by nebulized or aerosolized preparations.

In another embodiment of the invention, the active compound formulation is administered to a subject at the site of injury, usually within 100 hours of when an injury occurs (e.g., within 6, 12, or 24 hours, inclusive, of the time of the injury).

In a further embodiment, ophthalmic compositions of the present invention are used to prevent or reduce damage to retinal and optic nerve head tissues, as well as to enhance functional recovery after damage to ocular tissues. Ophthalmic conditions that may be treated include, but are not limited to, retinopathies (including diabetic retinopathy and retrolental fibroplasia), macular degeneration, ocular ischemia, glaucoma. Other conditions to be treated with the methods of the invention include damage associated with injuries to ophthalmic tissues, such as ischemia reperfusion injuries, photochemical injuries, and injuries associated with ocular surgery, particularly injuries to the retina or optic nerve head by exposure to light or surgical instruments. The ophthalmic compositions may also be used as an adjunct to ophthalmic surgery, such as by vitreal or subconjunctival injection following ophthalmic surgery. The compounds may be used for acute treatment of temporary conditions, or may be administered chronically, especially in the case of degenerative disease. The ophthalmic compositions may also be used prophylactically, especially prior to ocular surgery or noninvasive ophthalmic procedures or other types of surgery.

### Duration and Levels of Administration

In a preferred embodiment of the method of the invention, the active compound is administered to a subject for an extended period of time to produce optimum axonal outgrowth. Sustained contact with the active compound can be achieved, for example, by repeated administration of the active compound(s) over a period of time, such as one week, several weeks, one month or longer. The pharmaceutically acceptable formulation used to administer the active compound(s) can also be formulated to provide sustained delivery of the active compound to a subject. For example, the formulation may deliver the active compound for at least one, two, three, or four weeks, inclusive, following initial administration to the subject. For example, a subject to be treated in accordance with the present invention is treated with the active compound for at least 30 days (either by repeated administration or by use of a sustained delivery system, or both).

Sustained delivery of the active compound can be demonstrated by, for example, the continued therapeutic effect of the active compound over time (such as sustained delivery of the agents can be demonstrated by continued axonal growth in CNS neurons in a subject). Alternatively, sustained delivery of the active compound may be demonstrated by detecting the presence of the active compounds in vivo over time.

Approaches for sustained delivery include use of a polymeric capsule, a minipump to deliver the formulation, a biodegradable implant, or implanted transgenic autologous cells (see U.S. Pat. No. 6,214,622). Implantable infusion pump systems (e.g., INFUSAID pumps (Towanda, Pa.)); see Zierski et al., 1988; Kanoff, 1994) and osmotic pumps (sold by Alza Corporation) are available commercially and otherwise known in the art. Another mode of administration is via an implantable, externally programmable infusion pump. Suitable infusion pump systems and reservoir systems are also described in, e.g., U.S. Pat. No. 5,368,562 and No. 4,731,058.

Antibodies and antigen-binding portions thereof, particularly human, humanized and chimeric antibodies and antigen-binding portions can often be administered less frequently than other types of therapeutics. For example, an effective amount of such an antibody can range from about 0.01 mg/kg to about 5 or 10 mg/kg, inclusive; administered daily, weekly, biweekly, monthly or less frequently.

### Treatment of Neurological Disorders

Elements of the nervous system subject to disorders which may be treated with the compounds and methods of the invention include the central, somatic, autonomic, sympathetic and parasympathetic components of the nervous system, neurosensory tissues within the eye, ear, nose, mouth or other organs, as well as glial tissues associated with neuronal cells and structures. Neurological disorders may be caused by an injury to a neuron, such as a mechanical injury or an injury due to a toxic compound, by the abnormal growth or development of a neuron, or by the misregulation, such as downregulation, of an activity of a neuron. Neurological disorders can detrimentally affect nervous system functions such as the sensory function (the ability to sense changes within the body and the outside environment); the integrative function (the ability to interpret the changes); and the motor function (the ability to respond to the interpretation by initiating an action such as a muscular contraction or glandular secretion).

Examples of neurological disorders include traumatic or toxic injuries to peripheral or cranial nerves, spinal cord or to the brain, cranial nerves, traumatic brain injury, stroke, cerebral aneurism, and spinal cord injury. Other neurological disorders include cognitive and neurodegenerative disorders such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, hereditary motor and sensory neuropathy (Charcot-Marie-Tooth disease), diabetic neuropathy, progressive supranuclear palsy, epilepsy, and Jakob-Creutzfieldt disease. Autonomic function disorders include hypertension and sleep disorders.

Also to be treated with compounds and methods of the invention are neuropsychiatric disorders such as depression, schizophrenia, schizoaffective disorder, Korsakoff s psychosis, mania, anxiety disorders, or phobic disorders, learning or memory disorders (such as amnesia and age-related memory loss), attention deficit disorder, dysthymic disorder, major depressive disorder, mania, obsessive-compulsive disorder, psychoactive substance use disorders, anxiety, phobias, panic disorder, bipolar affective disorder, psychogenic pain syndromes, and eating disorders. Other examples of neurological disorders include injuries to the nervous system due to an infectious disease (such as meningitis, high fevers of various etiologies, HIV, syphilis, or post-polio syndrome) and injuries to the nervous system due to electricity (including contact with electricity or lightning, and complications from electro-convulsive psychiatric therapy). Neurological disorders associated with ophthalmic conditions include retina and optic nerve damage, glaucoma and age related macular degeneration.

The developing brain is a target for neurotoxicity in the developing central nervous system through many stages of pregnancy as well as during infancy and early childhood, and the methods of the invention may be utilized in preventing or treating neurological deficits in embryos or fetuses in utero, in premature infants, or in children with need of such treatment, including those with neurological birth defects. Further neurological disorders include, for example, those listed in HARRISON'S PRINCIPLES OF INTERNAL MEDICINE (Braunwald et al., McGraw-Hill, 2001) and in the AMERICAN PSYCHIATRIC ASSOCIATION'S DIAGNOSTIC AND STATISTICAL MANUAL OF MENTAL DISORDERS DSM-IV (American Psychiatric Press, 2000).

### In Vitro Treatment of Neurons

Neurons derived from the central or peripheral nervous system can be contacted with the agents ex vivo to promote axonal outgrowth in vitro. Accordingly, neurons can be isolated from a subject and grown in vitro, using techniques well known in the art, and then treated in accordance with the present invention to modulate axonal outgrowth. Briefly, a neuronal culture can be obtained by allowing neurons to migrate out of fragments of neural tissue adhering to a suitable substrate (such as a culture dish) or by disaggregating the tissue, such as mechanically or enzymatically, to produce a suspension of neurons. For example, the enzymes trypsin, collagenase, elastase, hyaluronidase, DNase, pronase, dispase, or various combinations thereof can be used. Methods for isolating neuronal tissue and the disaggregation of tissue to obtain isolated cells are described in Freshney, CULTURE OF ANIMAL CELLS, A MANUAL OF BASIC TECHNIQUE, (3rd ed., 1994). Such cells can be subsequently contacted with the agents in amounts and for a duration of time as described above. Once modulation of axonal outgrowth has been achieved in the neurons, these cells can be re-administered to the subject, such as by implantation.

### Detecting Neural Regenerative Effect

The ability of an agent to promote neural regeneration in a subject may be assessed using any of a variety of known procedures and assays. For example, the ability of an agent to re-establish neural connectivity and/or function after an injury, may be determined histologically (either by slicing neuronal tissue and looking at neuronal branching, or by showing cytoplasmic transport of dyes). Agents may also be assessed by monitoring the ability of the agent to fully or partially restore the electroretinogram after damage to the neural retina or optic nerve; or to fully or partially restore a pupillary response to light in the damaged eye.

Other tests that may be used include standard tests of neurological function in human subjects or in animal models of spinal injury (such as standard reflex testing, urologic tests, urodynamic testing, tests for deep and superficial pain appreciation, propnoceptive placing of the hind limbs, ambulation, and evoked potential testing). In addition, nerve impulse conduction can be measured in a subject, such as by measuring conduct action potentials, as an indication of the production of a neurosalutary effect.

Animal models suitable for use in the assays of the present invention include the rat model of partial transaction (see Weidner et al., 2001), that tests how well a compound can enhance the survival and sprouting of the intact remaining fragment of an almost fully-transected cord. Accordingly, after administration of a candidate agent these animals may be evaluated for recovery of a certain function, such as how well the rats may manipulate food pellets with their forearms (to which the relevant cord had been cut 97%).

Another animal model suitable for use in the assays of the present invention includes the rat model of stroke (see Kawamata et al., 1997, for various tests that may be used to assess sensor motor function in the limbs as well as vestibulomotor function after an injury). Administration to these animals of the agents of the invention can be used to assess whether a given compound, route of administration, or dosage provides a neuroregenerative effect, such as increasing the level of function, or increasing the rate of regaining function or the degree of retention of function in the test animals.

Standard neurological evaluations used to assess progress in human patients after a stroke may also be used to evaluate the ability of an agent to produce a neurosalutary effect in a subject. Such standard neurological evaluations are routine in the medical arts, and are described in, for example, “Guide to Clinical Neurobiology” Edited by Mohr and Gautier (Churchill Livingstone Inc. 1995).

### Transmembrane Protein Tyrosine Phosphatases (PTPs)

Transmembrane PTPs form a large and diverse molecular family, and have a structure typical of transmembrane cell surface receptors (18, 19). PTPσ is a receptor type protein-tyrosine phosphatase that has been cloned and identified in mouse, in rat, and in human (Pulido et al., Proc. Nat. Acad. Sci. 92: 11686-11690, 1995; PubMed ID: 8524829). PTPσ and other PTPs in the LAR subfamily can act as receptors for heparan sulfate proteoglycans (HSPGs), and these PTPs are involved in axon guidance and synapse formation during development. In the adult, PTPσ gene disruption enhances regeneration in sciatic, facial and optic nerves.

PTPσ has two intracellular PTPase domains and an extracellular region having Ig-like and fibronectin type III-like domains. The specific location of the various domains of the PTPσ molecule are known in the art. Pulido et al., 92 PNAS 11686 (1995); PubMed ID: 8524829.

Amino acid and nucleotide sequences for human and mouse PTPσ are shown herein.

Human PTPsigma transcript variant 4, amino acid sequence:

(SEQ ID NO: 1)
MAPTWGPGMVSVVGPMGLLVVLLVGGCAAEEPPRFIKEPKDQIGVSGGVASFVCQATGDPKPRVTWNKKG
KKVNSQRFETIEFDESAGAVLRIQPLRTPRDENVYECVAQNSVGEITVHAKLTVLREDQLPSGFPNIDMG
PQLKVVERTRTATMLCAASGNPDPEITWFKDFLPVDPSASNGRIKQLRSGALQIESSEETDQGKYECVAT
NSAGVRYSSPANLYVRELREVRRVAPRFSILPMSHEIMPGGNVNITCVAVGSPMPYVKWMQGAEDLTPED
DMPVGRNVLELTDVKDSANYTCVAMSSLGVIEAVAQITVKSLPKAPGTPMVTENTATSITITWDSGNPDP
VSYYVIEYKSKSQDGPYQIKEDITTTRYSIGGLSPNSEYEIWVSAVNSIGQGPPSESVVTRTGEQAPASA
PRNVQARMLSATTMIVQWEEPVEPNGLIRGYRVYYTMEPEHPVGNWQKHNVDDSLLTTVGSLLEDETYTV
RVLAFTSVGDGPLSDPIQVKTQQGVPGQPMNLRAEARSETSITLSWSPPRQESIIKYELLFREGDHGREV
GRTFDPTTSYVVEDLKPNTEYAFRLAARSPQGLGAFTPVVRQRTLQSISPKNFKVKMIMKTSVLLSWEFP
DNYNSPTPYKIQYNGLTLDVDGRTTKKLITHLKPHTFYNFVLTNRGSSLGGLQQTVTAWTAFNLLNGKPS
VAPKPDADGFIMVYLPDGQSPVPVQSYFIVMVPLRKSRGGQFLTPLGSPEDMDLEELIQDISRLQRRSLR
HSRQLEVPRPYIAARFSVLPPTFHPGDQKQYGGFDNRGLEPGHRYVLFVLAVLQKSEPTFAASPFSDPFQ
LDNPDPQPIVDGEEGLIWVIGPVLAVVFIICIVIAILLYKNKPDSKRKDSEPRTKCLLNNADLAPHHPKD
PVEMRRINFQTPGMLSHPPIPIADMAEHTERLKANDSLKLSQEYESIDPGQQFTWEHSNLEVNKPKNRYA
NVIAYDHSRVILQPIEGIMGSDYINANYVDGYRCQNAYIATQGPLPETFGDFWRMVWEQRSATIVMMTRL
EEKSRIKCDQYWPNRGTETYGFIQVTLLDTIELATFCVRTFSLHKNGSSEKREVRQFQFTAWPDHGVPEY
PTPFLAFLRRVKTCNPPDAGPIVVHCSAGVGRTGCFIVIDAMLERIKPEKTVDVYGHVTLMRSQRNYMVQ
TEDQYSFIHEALLEAVGCGNTEVPARSLYAYIQKLAQVEPGEHVTGMELEFKRLANSKAHTSRFISANLP
CNKFKNRLVNIMPYESTRVCLQPIRGVEGSDYINASFIDGYRQQKAYIATQGPLAETTEDFWRMLWENNS
TIVVMLTKLREMGREKCHQYWPAERSARYQYFVVDPMAEYNMPQYILREFKVTDARDGQSRTVRQFQFTD
WPEQGVPKSGEGFIDFIGQVHKTKEQFGQDGPISVHCSAGVGRTGVFITLSIVLERMRYEGVVDIFQTVK
MLRTQRPAMVQTEDEYQFCYQAALEYLGSFDHYAT

Human PTPsigma transcript variant 4, nucleotide sequence:

| (SEQ ID NO: 2) | |
|---|---|
| 1 | cctcgcgccg cccgcccggc agcccggccg gcgcgcgcac gccgcgagcc gctggcgctc |
| 61 | gggctccgct cggatcccat gcaacagcca cgatgtgaag cggggcagag ccgggggagc |
| 121 | ccagcccagc cagcctccag acgttgcccc atctgacgct cggctcgagg cctctctgtg |
| 181 | agggaccggg gggccatccc cctccagggc ggagatcgga ggtcgctgcc aagcatggcg |
| 241 | cccacctggg gccctggcat ggtgtctgtg gttggtccca tgggcctcct tgtggtcctg |
| 301 | ctcgttggag gctgtgcagc agaagagccc cccaggttta tcaaagaacc caaggaccag |
| 361 | atcggcgtgt cggggggtgt ggcctctttc gtgtgtcagg ccacgggtga ccccaagcca |
| 421 | cgagtgacct ggaacaagaa gggcaagaag gtcaactctc agcgctttga gacgattgag |
| 481 | tttgatgaga gtgcaggggc agtgctgagg atccagccgc tgaggacacc gcgggatgaa |
| 541 | aacgtgtacg agtgtgtggc ccagaactcg gttggggaga tcacagtcca tgccaagctt |
| 601 | actgtcctcc gagaggacca gctgccctct ggcttcccca acatcgacat gggcccacag |
| 661 | ttgaaggtgg tggagcggac acggacagcc accatgctct gtgcagccag cggcaaccct |
| 721 | gaccctgaga tcacctggtt caaggacttc ctgcctgtgg atcctagtgc cagcaatgga |
| 781 | cgcatcaaac agctgcgatc aggagccctg cagattgaaa gcagtgagga aaccgaccag |
| 841 | ggcaaatatg agtgtgtggc caccaacagc gccggcgtgc gctactcctc acctgccaac |
| 901 | ctctacgtgc gagagcttcg agaagtccgc cgcgtggccc cgcgcttctc catcctgccc |
| 961 | atgagccacg agatcatgcc agggggcaac gtgaacatca cctgcgtggc cgtgggctcg |
| 1021 | cccatgccat acgtgaagtg gatgcagggg gccgaggacc tgacccccga ggatgacatg |
| 1081 | cccgtgggtc ggaacgtgct ggaactcaca gatgtcaagg actcggccaa ctacacctgc |
| 1141 | gtggccatgt ccagcctggg cgtcattgag gcggttgctc agatcacggt gaaatctctc |
| 1201 | cccaaagctc ccgggactcc catggtgact gagaacacag ccaccagcat caccatcacg |
| 1261 | tgggactcgg gcaacccaga tcctgtgtcc tattacgtca tcgaatataa atccaagagc |
| 1321 | caagacgggc cgtatcagat taaagaggac atcaccacca cacgttacag catcggcggc |
| 1381 | ctgagcccca actcggagta cgagatctgg gtgtcggccg tcaactccat cggccagggg |
| 1441 | ccccccagcg agtccgtggt cacccgcaca ggcgagcagg ccccggccag cgcgccgcgg |
| 1501 | aacgtgcaag cccggatgct cagcgcgacc accatgattg tgcagtggga ggagccggtg |
| 1561 | gagcccaacg gcctgatccg cggctaccgc gtctactaca ccatggaacc ggagcacccc |
| 1621 | gtgggcaact ggcagaagca caacgtggac gacagcctgc tgaccaccgt gggcagcctg |
| 1681 | ctggaggacg agacctacac cgtgcgggtg ctcgccttca cctccgtcgg cgacgggccc |
| 1741 | ctctcggacc ccatccaggt caagacgcag cagggagtgc cgggccagcc catgaacctg |
| 1801 | cgggccgagg ccaggtcgga gaccagcatc acgctgtcct ggagcccccc gcggcaggag |
| 1861 | agtatcatca agtacgagct cctcttccgg gaaggcgacc atggccggga ggtgggaagg |
| 1921 | accttcgacc cgacgacttc ctacgtggtg gaggacctga agcccaacac ggagtacgcc |
| 1981 | ttccgcctgg cggcccgctc gccgcagggc ctgggcgcct tcacccccgt ggtgcggcag |
| 2041 | cgcacgctgc agtccatctc gcccaagaac ttcaaggtga aaatgatcat gaagacatca |
| 2101 | gttctgctca gctgggagtt ccctgacaac tacaactcac ccacacccta caagatccag |
| 2161 | tacaatgggc tcacactgga tgtggatggc cgtaccacca agaagctcat cacgcacctc |
| 2221 | aagccccaca ccttctacaa ctttgtgctg accaatcgcg gcagcagcct gggcggcctc |
| 2281 | cagcagacgg tcaccgcctg gactgccttc aacctgctca acggcaagcc cagcgtcgcc |
| 2341 | cccaagcctg atgctgacgg cttcatcatg gtgtatcttc ctgacggcca gagccccgtg |
| 2401 | cctgtccaga gctatttcat tgtgatggtg ccactgcgca agtctcgtgg aggccaattc |
| 2461 | ctgaccccgc tgggtagccc agaggacatg gatctggaag agctcatcca ggacatctca |
| 2521 | cggctacaga ggcgcagcct gcggcactcg cgtcagctgg aggtgccccg gccctatatt |
| 2581 | gcagctcgct tctctgtgct gccacccacg ttccatcccg gcgaccagaa gcagtatggc |
| 2641 | ggcttcgata accggggcct ggagcccggc caccgctatg tcctcttcgt gcttgccgtg |
| 2701 | cttcagaaga gcgagcctac ctttgcagcc agtcccttct cagacccctt ccagctggat |
| 2761 | aacccggacc cccagcccat cgtggatggc gaggaggggc ttatctgggt gatcgggcct |
| 2821 | gtgctggccg tggtcttcat aatctgcatt gtcattgcta tcctgctcta caagaacaaa |
| 2881 | cccgacagta aacgcaagga ctcagaaccc cgcaccaaat gcctcctgaa caatgccgac |
| 2941 | ctcgcccctc accaccccaa ggaccctgtg gaaatgagac gcattaactt ccagactcca |
| 3001 | ggcatgctta gccacccgcc aattcccatc gcagacatgg cggagcacac ggagcggctc |
| 3061 | aaggccaacg acagcctcaa gctctcccag gagtatgagt ccatcgaccc tggacagcag |
| 3121 | ttcacatggg aacattccaa cctggaagtg aacaagccga agaaccgcta tgccaacgtc |
| 3181 | atcgcctatg accactcccg tgtcatcctc cagcccattg aaggcatcat gggcagtgat |
| 3241 | tacatcaatg ccaactacgt ggacggctac cggtgtcaga acgcgtacat tgccacgcag |
| 3301 | gggccgctgc ctgagacctt tggggacttc tggcgtatgg tgtgggagca gcggtcggcg |
| 3361 | accatcgtca tgatgacgcg gctggaggag aagtcacgga tcaagtgtga tcagtattgg |
| 3421 | cccaacagag gcacggagac ctacggcttc atccaggtca cgttgctaga taccatcgag |
| 3481 | ctggccacat tctgcgtcag gacattctct ctgcacaaga atggctccag tgagaaacgc |
| 3541 | gaggtccgcc agttccagtt tacggcgtgg ccggaccatg gcgtgcccga atacccaacg |
| 3601 | cccttcctgg ctttcctgcg gagagtcaag acctgcaacc cgccagatgc cggccccatc |
| 3661 | gtggttcact gcagtgccgg tgtgggccgc acaggctgct ttatcgtcat cgacgccatg |
| 3721 | cttgagcgga tcaagccaga gaagacagtc gatgtctatg gccacgtgac gctcatgagg |
| 3781 | tcccagcgca actacatggt gcagacggag gaccagtaca gcttcatcca cgaggccctg |
| 3841 | ctggaggccg tgggctgtgg caacacagaa gtgcccgcac gcagcctcta tgcctacatc |
| 3901 | cagaagctgg cccaggtgga gcctggcgaa cacgtcactg gcatggaact cgagttcaag |
| 3961 | cggctggcta actccaaggc ccacacgtca cgcttcatca gtgccaatct gccttgtaac |
| 4021 | aagttcaaga accgcctggt gaacatcatg ccctatgaga gcacacgggt ctgtctgcaa |
| 4081 | cccatccggg gtgtggaggg ctctgactac atcaacgcca gcttcattga tggctacagg |
| 4141 | cagcagaagg cctacatcgc gacacagggg ccgctggcgg agaccacgga agacttctgg |
| 4201 | cgcatgctgt gggagaacaa ttcgacgatc gtggtgatgc tgaccaagct gcgggagatg |
| 4261 | ggccgggaga agtgtcacca gtactggccg gccgagcgct ctgcccgcta ccagtacttt |
| 4321 | gtggtagatc cgatggcaga atacaacatg cctcagtata tcctgcgaga gttcaaggtc |
| 4381 | acagatgccc gggatggcca gtcccggact gtccggcagt tccagttcac agactggccg |
| 4441 | gaacagggtg tgccaaagtc gggggagggc ttcatcgact tcattggcca agtgcataag |
| 4501 | actaaggagc agtttggcca ggacggcccc atctctgtcc actgcagtgc cggcgtgggc |
| 4561 | aggacgggcg tcttcatcac gcttagcatc gtgctggagc ggatgcggta tgaaggcgtg |
| 4621 | gtggacatct ttcagacggt gaagatgcta cgaacccagc ggccggccat ggtgcagaca |
| 4681 | gaggatgagt accagttctg ttaccaggcg gcactggagt acctcggaag ctttgaccac |
| 4741 | tatgcaacct aaagccatgg ttccccccag gcccgacacc actggccccg gatgcctctg |
| 4801 | cccctcccgg gcggacctcc tgaggcctgg acccccagtg ggcagggcag gaggtggcag |
| 4861 | cggcagcagc tgtgtttctg caccatttcc gaggacgacg cagcccctcg agccccccca |
| 4921 | ccggccccgg ccgccccagc gacctccctg gcaccggccg ccgccttcaa atacttggca |
| 4981 | cattcctcct ttccttccaa ttccaaaacc agattccggg gtggggggtg gggggatggt |
| 5041 | gagcaaatag gagtgctccc cagaaccaga ggagggtggg gcacagacca tagacggacc |
| 5101 | cctcgtcctc ccccagcggt ggtaggggga cccggggggc tcctccccgc tctgcagcct |
| 5161 | ggggacactg ggctgggacc agaatccagc tttcttttaa aactctcagt gtaactgtat |
| 5221 | cccgtgacat ttcatttttt ttaaatagtg tatttttttt tccatttttt tttttaagag |
| 5281 | aaacaaacaa aagactcgcc agtcaatgac tttcaaagag aactaacttt ggcttattca |
| 5341 | tattctgttc aaagacagtc tattttttca ctgtagaaag cgtccttgtg tgatagttac |
| 5401 | gttcgcaaac gcgcacgcca ggcccatggc tgtaccttgg cttttttttt tttttttttt |
| 5461 | tttttaattt ttcctaccat cagaaagtgt gctttgctca cagaagaatg ggatgtcctt |
| 5521 | ttttctttct tggctttttt tttccccctt tttgtttcat ttttataaat taaattttca |
| 5581 | gacatatcaa atacagttct gagggtaagg tcatggggga gctcggaccc agtggcgttg |
| 5641 | ggtgcggttg agggggacgc tgctgtaaga ggagagagat gacagtggtc ctcctctgag |
| 5701 | agcctgagct gtctccccgt ctcccgcccc caaggagaca gagaggatcc tacttcttcg |
| 5761 | gggacagtgg ctgtatggct gtgctgcccc acatcaggga ccctttcccc ctgggactgt |
| 5821 | ggggcagttt gggagcaaaa ccagaaggac aggcccccct ctacccgcct accctgagca |
| 5881 | agcgagttgt tcctctttgt acaagggcag gtctgcggtt actttcaaca ctgtttattc |
| 5941 | cagcggaagc agccgggtgg ttttcccacc cccgtgtatg tagatatatc gactttgtat |
| 6001 | taaaggaaga tcgtctga |

Human PTPσ transcript variant 3 amino acid sequence:

(SEQ ID NO: 3)
MAPTWGPGMVSVVGPMGLLVVLLVGGCAAEEPPRFIKEPKDQIGVSGGVASFVCQATGDPKPRVTWNKKG
KKVNSQRFETIEFDESAGAVLRIQPLRTPRDENVYECVAQNSVGEITVHAKLTVLREDQLPSGFPNIDMG
PQLKVVERTRTATMLCAASGNPDPEITWFKDFLPVDPSASNGRIKQLRSGALQIESSEETDQGKYECVAT
NSAGVRYSSPANLYVRVRRVAPRFSILPMSHEIMPGGNVNITCVAVGSPMPYVKWMQGAEDLTPEDDMPV
GRNVLELTDVKDSANYTCVAMSSLGVIEAVAQITVKSLPKAPGTPMVTENTATSITITWDSGNPDPVSYY
VIEYKSKSQDGPYQIKEDITTTRYSIGGLSPNSEYEIWVSAVNSIGQGPPSESVVTRTGEQAPASAPRNV
QARMLSATTMIVQWEEPVEPNGLIRGYRVYYTMEPEHPVGNWQKHNVDDSLLTTVGSLLEDETYTVRVLA
FTSVGDGPLSDPIQVKTQQGVPGQPMNLRAEARSETSITLSWSPPRQESIIKYELLFREGDHGREVGRTF
DPTTSYVVEDLKPNTEYAFRLAARSPQGLGAFTPVVRQRTLQSISPKNFKVKMIMKTSVLLSWEFPDNYN
SPTPYKIQYNGLTLDVDGRTTKKLITHLKPHTFYNFVLTNRGSSLGGLQQTVTAWTAFNLLNGKPSVAPK
PDADGFIMVYLPDGQSPVPVQSYFIVMVPLRKSRGGQFLTPLGSPEDMDLEELIQDISRLQRRSLRHSRQ
LEVPRPYIAARFSVLPPTFHPGDQKQYGGFDNRGLEPGHRYVLFVLAVLQKSEPTFAASPFSDPFQLDNP
DPQPIVDGEEGLIWVIGPVLAVVFIICIVIAILLYKNKPDSKRKDSEPRTKCLLNNADLAPHHPKDPVEM
RRINFQTPGMLSHPPIPIADMAEHTERLKANDSLKLSQEYESIDPGQQFTWEHSNLEVNKPKNRYANVIA
YDHSRVILQPIEGIMGSDYINANYVDGYRCQNAYIATQGPLPETFGDFWRMVWEQRSATIVMMTRLEEKS
RIKCDQYWPNRGTETYGFIQVTLLDTIELATFCVRTFSLHKNGSSEKREVRQFQFTAWPDHGVPEYPTPF
LAFLRRVKTCNPPDAGPIVVHCSAGVGRTGCFIVIDAMLERIKPEKTVDVYGHVTLMRSQRNYMVQTEDQ
YSFIHEALLEAVGCGNTEVPARSLYAYIQKLAQVEPGEHVTGMELEFKRLANSKAHTSRFISANLPCNKF
KNRLVNIMPYESTRVCLQPIRGVEGSDYINASFIDGYRQQKAYIATQGPLAETTEDFWRMLWENNSTIVV
MLTKLREMGREKCHQYWPAERSARYQYFVVDPMAEYNMPQYILREFKVTDARDGQSRTVRQFQFTDWPEQ
GVPKSGEGFIDFIGQVHKTKEQFGQDGPISVHCSAGVGRTGVFITLSIVLERMRYEGVVDIFQTVKMLRT
QRPAMVQTEDEYQFCYQAALEYLGSFDHYAT

Human PTPσ transcript variant 3 nucleotide sequence:

| (SEQ ID NO: 4) | |
|---|---|
| 1 | cctcgcgccg cccgcccggc agcccggccg gcgcgcgcac gccgcgagcc gctggcgctc |
| 61 | gggctccgct cggatcccat gcaacagcca cgatgtgaag cggggcagag ccgggggagc |
| 121 | ccagcccagc cagcctccag acgttgcccc atctgacgct cggctcgagg cctctctgtg |
| 181 | agggaccggg gggccatccc cctccagggc ggagatcgga ggtcgctgcc aagcatggcg |
| 241 | cccacctggg gccctggcat ggtgtctgtg gttggtccca tgggcctcct tgtggtcctg |
| 301 | ctcgttggag gctgtgcagc agaagagccc cccaggttta tcaaagaacc caaggaccag |
| 361 | atcggcgtgt cggggggtgt ggcctctttc gtgtgtcagg ccacgggtga ccccaagcca |
| 421 | cgagtgacct ggaacaagaa gggcaagaag gtcaactctc agcgctttga gacgattgag |
| 481 | tttgatgaga gtgcaggggc agtgctgagg atccagccgc tgaggacacc gcgggatgaa |
| 541 | aacgtgtacg agtgtgtggc ccagaactcg gttggggaga tcacagtcca tgccaagctt |
| 601 | actgtcctcc gagaggacca gctgccctct ggcttcccca acatcgacat gggcccacag |
| 661 | ttgaaggtgg tggagcggac acggacagcc accatgctct gtgcagccag cggcaaccct |
| 721 | gaccctgaga tcacctggtt caaggacttc ctgcctgtgg atcctagtgc cagcaatgga |
| 781 | cgcatcaaac agctgcgatc aggagccctg cagattgaaa gcagtgagga aaccgaccag |
| 841 | ggcaaatatg agtgtgtggc caccaacagc gccggcgtgc gctactcctc acctgccaac |
| 901 | ctctacgtgc gagtccgccg cgtggccccg cgcttctcca tcctgcccat gagccacgag |
| 961 | atcatgccag ggggcaacgt gaacatcacc tgcgtggccg tgggctcgcc catgccatac |
| 1021 | gtgaagtgga tgcagggggc cgaggacctg acccccgagg atgacatgcc cgtgggtcgg |
| 1081 | aacgtgctgg aactcacaga tgtcaaggac tcggccaact acacctgcgt ggccatgtcc |
| 1141 | agcctgggcg tcattgaggc ggttgctcag atcacggtga aatctctccc caaagctccc |
| 1201 | gggactccca tggtgactga gaacacagcc accagcatca ccatcacgtg ggactcgggc |
| 1261 | aacccagatc ctgtgtccta ttacgtcatc gaatataaat ccaagagcca agacgggccg |
| 1321 | tatcagatta aagaggacat caccaccaca cgttacagca tcggcggcct gagccccaac |
| 1381 | tcggagtacg agatctgggt gtcggccgtc aactccatcg gccaggggcc ccccagcgag |
| 1441 | tccgtggtca cccgcacagg cgagcaggcc ccggccagcg cgccgcggaa cgtgcaagcc |
| 1501 | cggatgctca gcgcgaccac catgattgtg cagtgggagg agccggtgga gcccaacggc |
| 1561 | ctgatccgcg gctaccgcgt ctactacacc atggaaccgg agcaccccgt gggcaactgg |
| 1621 | cagaagcaca acgtggacga cagcctgctg accaccgtgg gcagcctgct ggaggacgag |
| 1681 | acctacaccg tgcgggtgct cgccttcacc tccgtcggcg acgggcccct ctcggacccc |
| 1741 | atccaggtca agacgcagca gggagtgccg ggccagccca tgaacctgcg ggccgaggcc |
| 1801 | aggtcggaga ccagcatcac gctgtcctgg agccccccgc ggcaggagag tatcatcaag |
| 1861 | tacgagctcc tcttccggga aggcgaccat ggccgggagg tgggaaggac cttcgacccg |
| 1921 | acgacttcct acgtggtgga ggacctgaag cccaacacgg agtacgcctt ccgcctggcg |
| 1981 | gcccgctcgc cgcagggcct gggcgccttc acccccgtgg tgcggcagcg cacgctgcag |
| 2041 | tccatctcgc ccaagaactt caaggtgaaa atgatcatga agacatcagt tctgctcagc |
| 2101 | tgggagttcc ctgacaacta caactcaccc acaccctaca agatccagta caatgggctc |
| 2161 | acactggatg tggatggccg taccaccaag aagctcatca cgcacctcaa gccccacacc |
| 2221 | ttctacaact ttgtgctgac caatcgcggc agcagcctgg gcggcctcca gcagacggtc |
| 2281 | accgcctgga ctgccttcaa cctgctcaac ggcaagccca gcgtcgcccc caagcctgat |
| 2341 | gctgacggct tcatcatggt gtatcttcct gacggccaga gccccgtgcc tgtccagagc |
| 2401 | tatttcattg tgatggtgcc actgcgcaag tctcgtggag gccaattcct gaccccgctg |
| 2461 | ggtagcccag aggacatgga tctggaagag ctcatccagg acatctcacg gctacagagg |
| 2521 | cgcagcctgc ggcactcgcg tcagctggag gtgccccggc cctatattgc agctcgcttc |
| 2581 | tctgtgctgc cacccacgtt ccatcccggc gaccagaagc agtatggcgg cttcgataac |
| 2641 | cggggcctgg agcccggcca ccgctatgtc ctcttcgtgc ttgccgtgct tcagaagagc |
| 2701 | gagcctacct ttgcagccag tcccttctca gaccccttcc agctggataa cccggacccc |
| 2761 | cagcccatcg tggatggcga ggaggggctt atctgggtga tcgggcctgt gctggccgtg |
| 2821 | gtcttcataa tctgcattgt cattgctatc ctgctctaca agaacaaacc cgacagtaaa |
| 2881 | cgcaaggact cagaaccccg caccaaatgc ctcctgaaca atgccgacct cgcccctcac |
| 2941 | caccccaagg accctgtgga aatgagacgc attaacttcc agactccagg catgcttagc |
| 3001 | cacccgccaa ttcccatcgc agacatggcg gagcacacgg agcggctcaa ggccaacgac |
| 3061 | agcctcaagc tctcccagga gtatgagtcc atcgaccctg gacagcagtt cacatgggaa |
| 3121 | cattccaacc tggaagtgaa caagccgaag aaccgctatg ccaacgtcat cgcctatgac |
| 3181 | cactcccgtg tcatcctcca gcccattgaa ggcatcatgg gcagtgatta catcaatgcc |
| 3241 | aactacgtgg acggctaccg gtgtcagaac gcgtacattg ccacgcaggg gccgctgcct |
| 3301 | gagacctttg gggacttctg gcgtatggtg tgggagcagc ggtcggcgac catcgtcatg |
| 3361 | atgacgcggc tggaggagaa gtcacggatc aagtgtgatc agtattggcc caacagaggc |
| 3421 | acggagacct acggcttcat ccaggtcacg ttgctagata ccatcgagct ggccacattc |
| 3481 | tgcgtcagga cattctctct gcacaagaat ggctccagtg agaaacgcga ggtccgccag |
| 3541 | ttccagttta cggcgtggcc ggaccatggc gtgcccgaat acccaacgcc cttcctggct |
| 3601 | ttcctgcgga gagtcaagac ctgcaacccg ccagatgccg gccccatcgt ggttcactgc |
| 3661 | agtgccggtg tgggccgcac aggctgcttt atcgtcatcg acgccatgct tgagcggatc |
| 3721 | aagccagaga agacagtcga tgtctatggc cacgtgacgc tcatgaggtc ccagcgcaac |
| 3781 | tacatggtgc agacggagga ccagtacagc ttcatccacg aggccctgct ggaggccgtg |
| 3841 | ggctgtggca acacagaagt gcccgcacgc agcctctatg cctacatcca gaagctggcc |
| 3901 | caggtggagc ctggcgaaca cgtcactggc atggaactcg agttcaagcg gctggctaac |
| 3961 | tccaaggccc acacgtcacg cttcatcagt gccaatctgc cttgtaacaa gttcaagaac |
| 4021 | cgcctggtga acatcatgcc ctatgagagc acacgggtct gtctgcaacc catccggggt |
| 4081 | gtggagggct ctgactacat caacgccagc ttcattgatg gctacaggca gcagaaggcc |
| 4141 | tacatcgcga cacaggggcc gctggcggag accacggaag acttctggcg catgctgtgg |
| 4201 | gagaacaatt cgacgatcgt ggtgatgctg accaagctgc gggagatggg ccgggagaag |
| 4261 | tgtcaccagt actggccggc cgagcgctct gcccgctacc agtactttgt ggtagatccg |
| 4321 | atggcagaat acaacatgcc tcagtatatc ctgcgagagt tcaaggtcac agatgcccgg |
| 4381 | gatggccagt cccggactgt ccggcagttc cagttcacag actggccgga acagggtgtg |
| 4441 | ccaaagtcgg gggagggctt catcgacttc attggccaag tgcataagac taaggagcag |
| 4501 | tttggccagg acggccccat ctctgtccac tgcagtgccg gcgtgggcag gacgggcgtc |
| 4561 | ttcatcacgc ttagcatcgt gctggagcgg atgcggtatg aaggcgtggt ggacatcttt |
| 4621 | cagacggtga agatgctacg aacccagcgg ccggccatgg tgcagacaga ggatgagtac |
| 4681 | cagttctgtt accaggcggc actggagtac ctcggaagct ttgaccacta tgcaacctaa |
| 4741 | agccatggtt ccccccaggc ccgacaccac tggccccgga tgcctctgcc cctcccgggc |
| 4801 | ggacctcctg aggcctggac ccccagtggg cagggcagga ggtggcagcg gcagcagctg |
| 4861 | tgtttctgca ccatttccga ggacgacgca gcccctcgag cccccccacc ggccccggcc |
| 4921 | gccccagcga cctccctggc accggccgcc gccttcaaat acttggcaca ttcctccttt |
| 4981 | ccttccaatt ccaaaaccag attccggggt ggggggtggg gggatggtga gcaaatagga |
| 5041 | gtgctcccca gaaccagagg agggtggggc acagaccata gacggacccc tcgtcctccc |
| 5101 | ccagcggtgg tagggggacc cggggggctc ctccccgctc tgcagcctgg ggacactggg |
| 5161 | ctgggaccag aatccagctt tcttttaaaa ctctcagtgt aactgtatcc cgtgacattt |
| 5221 | catttttttt aaatagtgta tttttttttc catttttttt tttaagagaa acaaacaaaa |
| 5281 | gactcgccag tcaatgactt tcaaagagaa ctaactttgg cttattcata ttctgttcaa |
| 5341 | agacagtcta ttttttcact gtagaaagcg tccttgtgtg atagttacgt tcgcaaacgc |
| 5401 | gcacgccagg cccatggctg taccttggct tttttttttt tttttttttt tttaattttt |
| 5461 | cctaccatca gaaagtgtgc tttgctcaca gaagaatggg atgtcctttt ttctttcttg |
| 5521 | gctttttttt tccccctttt tgtttcattt ttataaatta aattttcaga catatcaaat |
| 5581 | acagttctga gggtaaggtc atgggggagc tcggacccag tggcgttggg tgcggttgag |
| 5641 | ggggacgctg ctgtaagagg agagagatga cagtggtcct cctctgagag cctgagctgt |
| 5701 | ctccccgtct cccgccccca aggagacaga gaggatccta cttcttcggg gacagtggct |
| 5761 | gtatggctgt gctgccccac atcagggacc ctttccccct gggactgtgg ggcagtttgg |
| 5821 | gagcaaaacc agaaggacag gcccccctct acccgcctac cctgagcaag cgagttgttc |
| 5881 | ctctttgtac aagggcaggt ctgcggttac tttcaacact gtttattcca gcggaagcag |
| 5941 | ccgggtggtt ttcccacccc cgtgtatgta gatatatcga ctttgtatta aaggaagatc |
| 6001 | gtctga |

Human PTPσ transcript variant 1 amino acid sequence:

(SEQ ID NO: 5)
MAPTWGPGMVSVVGPMGLLVVLLVGGCAAEEPPRFIKEPKDQIGVSGGVASFVCQATGDPKPRVTWNKKG
KKVNSQRFETIEFDESAGAVLRIQPLRTPRDENVYECVAQNSVGEITVHAKLTVLREDQLPSGFPNIDMG
PQLKVVERTRTATMLCAASGNPDPEITWFKDFLPVDPSASNGRIKQLRSETFESTPIRGALQIESSEETD
QGKYECVATNSAGVRYSSPANLYVRELREVRRVAPRFSILPMSHEIMPGGNVNITCVAVGSPMPYVKWMQ
GAEDLTPEDDMPVGRNVLELTDVKDSANYTCVAMSSLGVIEAVAQITVKSLPKAPGTPMVTENTATSITI
TWDSGNPDPVSYYVIEYKSKSQDGPYQIKEDITTTRYSIGGLSPNSEYEIWVSAVNSIGQGPPSESVVTR
TGEQAPASAPRNVQARMLSATTMIVQWEEPVEPNGLIRGYRVYYTMEPEHPVGNWQKHNVDDSLLTTVGS
LLEDETYTVRVLAFTSVGDGPLSDPIQVKTQQGVPGQPMNLRAEARSETSITLSWSPPRQESIIKYELLF
REGDHGREVGRTFDPTTSYVVEDLKPNTEYAFRLAARSPQGLGAFTPVVRQRTLQSKPSAPPQDVKCVSV
RSTAILVSWRPPPPETHNGALVGYSVRYRPLGSEDPEPKEVNGIPPTTTQILLEALEKWTQYRITTVAHT
EVGPGPESSPVVVRTDEDVPSAPPRKVEAEALNATAIRVLWRSPAPGRQHGQIRGYQVHYVRMEGAEARG
PPRIKDVMLADAQWETDDTAEYEMVITNLQPETAYSITVAAYTMKGDGARSKPKVVVTKGAVLGRPTLSV
QQTPEGSLLARWEPPAGTAEDQVLGYRLQFGREDSTPLATLEFPPSEDRYTASGVHKGATYVFRLAARSR
GGLGEEAAEVLSIPEDTPRGHPQILEAAGNASAGTVLLRWLPPVPAERNGAIVKYTVAVREAGALGPARE
TELPAAAEPGAENALTLQGLKPDTAYDLQVRAHTRRGPGPFSPPVRYRTFLRDQVSPKNFKVKMIMKTSV
LLSWEFPDNYNSPTPYKIQYNGLTLDVDGRTTKKLITHLKPHTFYNFVLTNRGSSLGGLQQTVTAWTAFN
LLNGKPSVAPKPDADGFIMVYLPDGQSPVPVQSYFIVMVPLRKSRGGQFLTPLGSPEDMDLEELIQDISR
LQRRSLRHSRQLEVPRPYIAARFSVLPPTFHPGDQKQYGGFDNRGLEPGHRYVLFVLAVLQKSEPTFAAS
PFSDPFQLDNPDPQPIVDGEEGLIWVIGPVLAVVFIICIVIAILLYKNKPDSKRKDSEPRTKCLLNNADL
APHHPKDPVEMRRINFQTPDSGLRSPLREPGFHFESMLSHPPIPIADMAEHTERLKANDSLKLSQEYESI
DPGQQFTWEHSNLEVNKPKNRYANVIAYDHSRVILQPIEGIMGSDYINANYVDGYRCQNAYIATQGPLPE
TFGDFWRMVWEQRSATIVMMTRLEEKSRIKCDQYWPNRGTETYGFIQVTLLDTIELATFCVRTFSLHKNG
SSEKREVRQFQFTAWPDHGVPEYPTPFLAFLRRVKTCNPPDAGPIVVHCSAGVGRTGCFIVIDAMLERIK
PEKTVDVYGHVTLMRSQRNYMVQTEDQYSFIHEALLEAVGCGNTEVPARSLYAYIQKLAQVEPGEHVTGM
ELEFKRLANSKAHTSRFISANLPCNKFKNRLVNIMPYESTRVCLQPIRGVEGSDYINASFIDGYRQQKAY
IATQGPLAETTEDFWRMLWENNSTIVVMLTKLREMGREKCHQYWPAERSARYQYFVVDPMAEYNMPQYIL
REFKVTDARDGQSRTVRQFQFTDWPEQGVPKSGEGFIDFIGQVHKTKEQFGQDGPISVHCSAGVGRTGVF
ITLSIVLERMRYEGVVDIFQTVKMLRTQRPAMVQTEDEYQFCYQAALEYLGSFDHYAT

Human PTPσ transcript variant 1 nucleotide sequence:

| (SEQ ID NO: 6) | |
|---|---|
| 1 | cctcgcgccg cccgcccggc agcccggccg gcgcgcgcac gccgcgagcc gctggcgctc |
| 61 | gggctccgct cggatcccat gcaacagcca cgatgtgaag cggggcagag ccgggggagc |
| 121 | ccagcccagc cagcctccag acgttgcccc atctgacgct cggctcgagg cctctctgtg |
| 181 | agggaccggg gggccatccc cctccagggc ggagatcgga ggtcgctgcc aagcatggcg |
| 241 | cccacctggg gccctggcat ggtgtctgtg gttggtccca tgggcctcct tgtggtcctg |
| 301 | ctcgttggag gctgtgcagc agaagagccc cccaggttta tcaaagaacc caaggaccag |
| 361 | atcggcgtgt cggggggtgt ggcctctttc gtgtgtcagg ccacgggtga ccccaagcca |
| 421 | cgagtgacct ggaacaagaa gggcaagaag gtcaactctc agcgctttga gacgattgag |
| 481 | tttgatgaga gtgcaggggc agtgctgagg atccagccgc tgaggacacc gcgggatgaa |
| 541 | aacgtgtacg agtgtgtggc ccagaactcg gttggggaga tcacagtcca tgccaagctt |
| 601 | actgtcctcc gagaggacca gctgccctct ggcttcccca acatcgacat gggcccacag |
| 661 | ttgaaggtgg tggagcggac acggacagcc accatgctct gtgcagccag cggcaaccct |
| 721 | gaccctgaga tcacctggtt caaggacttc ctgcctgtgg atcctagtgc cagcaatgga |
| 781 | cgcatcaaac agctgcgatc agaaaccttt gaaagcactc cgattcgagg agccctgcag |
| 841 | attgaaagca gtgaggaaac cgaccagggc aaatatgagt gtgtggccac caacagcgcc |
| 901 | ggcgtgcgct actcctcacc tgccaacctc tacgtgcgag agcttcgaga agtccgccgc |
| 961 | gtggccccgc gcttctccat cctgcccatg agccacgaga tcatgccagg gggcaacgtg |
| 1021 | aacatcacct gcgtggccgt gggctcgccc atgccatacg tgaagtggat gcagggggcc |
| 1081 | gaggacctga cccccgagga tgacatgccc gtgggtcgga acgtgctgga actcacagat |
| 1141 | gtcaaggact cggccaacta cacctgcgtg gccatgtcca gcctgggcgt cattgaggcg |
| 1201 | gttgctcaga tcacggtgaa atctctcccc aaagctcccg ggactcccat ggtgactgag |
| 1261 | aacacagcca ccagcatcac catcacgtgg gactcgggca acccagatcc tgtgtcctat |
| 1321 | tacgtcatcg aatataaatc caagagccaa gacgggccgt atcagattaa agaggacatc |
| 1381 | accaccacac gttacagcat cggcggcctg agccccaact cggagtacga gatctgggtg |
| 1441 | tcggccgtca actccatcgg ccaggggccc cccagcgagt ccgtggtcac ccgcacaggc |
| 1501 | gagcaggccc cggccagcgc gccgcggaac gtgcaagccc ggatgctcag cgcgaccacc |
| 1561 | atgattgtgc agtgggagga gccggtggag cccaacggcc tgatccgcgg ctaccgcgtc |
| 1621 | tactacacca tggaaccgga gcaccccgtg ggcaactggc agaagcacaa cgtggacgac |
| 1681 | agcctgctga ccaccgtggg cagcctgctg gaggacgaga cctacaccgt gcgggtgctc |
| 1741 | gccttcacct ccgtcggcga cgggcccctc tcggacccca tccaggtcaa gacgcagcag |
| 1801 | ggagtgccgg gccagcccat gaacctgcgg gccgaggcca ggtcggagac cagcatcacg |
| 1861 | ctgtcctgga gccccccgcg gcaggagagt atcatcaagt acgagctcct cttccgggaa |
| 1921 | ggcgaccatg gccgggaggt gggaaggacc ttcgacccga cgacttccta cgtggtggag |
| 1981 | gacctgaagc ccaacacgga gtacgccttc cgcctggcgg cccgctcgcc gcagggcctg |
| 2041 | ggcgccttca cccccgtggt gcggcagcgc acgctgcagt ccaaaccgtc agccccccct |
| 2101 | caagacgtta aatgtgtcag cgtgcgctcc acggccattt tggtaagttg gcgcccgccg |
| 2161 | ccgccggaaa cgcacaacgg ggccctggtg ggctacagcg tccgctaccg accgctgggc |
| 2221 | tcagaggacc cggaacccaa ggaggtgaac ggcatccccc cgaccaccac tcagatcctg |
| 2281 | ctggaggcct tggagaagtg gacccagtac cgcatcacga ctgtcgctca cacagaggtg |
| 2341 | ggaccagggc ccgagagctc gcccgtggtc gtccgcaccg acgaggatgt gcccagcgcg |
| 2401 | ccgccgcgga aggtggaggc ggaggcgctc aacgccacgg ccatccgcgt gctgtggcgc |
| 2461 | tcgcccgcgc ccggccggca gcacggccag atccgcggct accaggtcca ctacgtgcgc |
| 2521 | atggagggcg ccgaggcccg cgggccgccg cgcatcaagg acgtcatgct ggccgatgcc |
| 2581 | cagtgggaga cggatgacac ggccgaatat gagatggtca tcacaaactt gcagcctgag |
| 2641 | accgcgtact ccatcacggt agccgcctac accatgaagg gcgatggcgc tcgcagcaaa |
| 2701 | cccaaggtgg ttgtgaccaa gggagcagtg ctgggccgcc caaccctgtc ggtgcagcag |
| 2761 | acccccgagg gcagcctgct ggcacgctgg gagcccccgg ctggcaccgc ggaggaccag |
| 2821 | gtgctgggct accgcctgca gtttggccgt gaggactcga cgcccctggc caccctggag |
| 2881 | ttcccgccct ccgaggaccg ctacacggca tcaggcgtgc acaagggggc cacgtatgtg |
| 2941 | ttccggcttg cggcccggag ccgcggcggc ctgggcgagg aggcagccga ggtcctgagc |
| 3001 | atcccggagg acacgccccg tggccacccg cagattctgg aggcggccgg caacgcctcg |
| 3061 | gccgggaccg tccttctccg ctggctgcca cccgtgcccg ccgagcgcaa cggggccatc |
| 3121 | gtcaaataca cggtggccgt gcgggaggcc ggtgccctgg gccctgcccg agagactgag |
| 3181 | ctgccggcag cggctgagcc gggcgcggag aacgcgctca cgctgcaggg cctgaagccc |
| 3241 | gacacggcct atgacctcca agtgcgagcc cacacgcgcc ggggccctgg ccccttcagc |
| 3301 | ccccccgtcc gctaccggac gttcctgcgg gaccaagtct cgcccaagaa cttcaaggtg |
| 3361 | aaaatgatca tgaagacatc agttctgctc agctgggagt tccctgacaa ctacaactca |
| 3421 | cccacaccct acaagatcca gtacaatggg ctcacactgg atgtggatgg ccgtaccacc |
| 3481 | aagaagctca tcacgcacct caagccccac accttctaca actttgtgct gaccaatcgc |
| 3541 | ggcagcagcc tgggcggcct ccagcagacg gtcaccgcct ggactgcctt caacctgctc |
| 3601 | aacggcaagc ccagcgtcgc ccccaagcct gatgctgacg gcttcatcat ggtgtatctt |
| 3661 | cctgacggcc agagccccgt gcctgtccag agctatttca ttgtgatggt gccactgcgc |
| 3721 | aagtctcgtg gaggccaatt cctgaccccg ctgggtagcc cagaggacat ggatctggaa |
| 3781 | gagctcatcc aggacatctc acggctacag aggcgcagcc tgcggcactc gcgtcagctg |
| 3841 | gaggtgcccc ggccctatat tgcagctcgc ttctctgtgc tgccacccac gttccatccc |
| 3901 | ggcgaccaga agcagtatgg cggcttcgat aaccggggcc tggagcccgg ccaccgctat |
| 3961 | gtcctcttcg tgcttgccgt gcttcagaag agcgagccta cctttgcagc cagtcccttc |
| 4021 | tcagacccct tccagctgga taacccggac ccccagccca tcgtggatgg cgaggagggg |
| 4081 | cttatctggg tgatcgggcc tgtgctggcc gtggtcttca taatctgcat tgtcattgct |
| 4141 | atcctgctct acaagaacaa acccgacagt aaacgcaagg actcagaacc ccgcaccaaa |
| 4201 | tgcctcctga acaatgccga cctcgcccct caccacccca aggaccctgt ggaaatgaga |
| 4261 | cgcattaact tccagactcc agattcaggc ctcaggagcc ccctcaggga gccggggttt |
| 4321 | cactttgaaa gcatgcttag ccacccgcca attcccatcg cagacatggc ggagcacacg |
| 4381 | gagcggctca aggccaacga cagcctcaag ctctcccagg agtatgagtc catcgaccct |
| 4441 | ggacagcagt tcacatggga acattccaac ctggaagtga acaagccgaa gaaccgctat |
| 4501 | gccaacgtca tcgcctatga ccactcccgt gtcatcctcc agcccattga aggcatcatg |
| 4561 | ggcagtgatt acatcaatgc caactacgtg gacggctacc ggtgtcagaa cgcgtacatt |
| 4621 | gccacgcagg ggccgctgcc tgagaccttt ggggacttct ggcgtatggt gtgggagcag |
| 4681 | cggtcggcga ccatcgtcat gatgacgcgg ctggaggaga agtcacggat caagtgtgat |
| 4741 | cagtattggc ccaacagagg cacggagacc tacggcttca tccaggtcac gttgctagat |
| 4801 | accatcgagc tggccacatt ctgcgtcagg acattctctc tgcacaagaa tggctccagt |
| 4861 | gagaaacgcg aggtccgcca gttccagttt acggcgtggc cggaccatgg cgtgcccgaa |
| 4921 | tacccaacgc ccttcctggc tttcctgcgg agagtcaaga cctgcaaccc gccagatgcc |
| 4981 | ggccccatcg tggttcactg cagtgccggt gtgggccgca caggctgctt tatcgtcatc |
| 5041 | gacgccatgc ttgagcggat caagccagag aagacagtcg atgtctatgg ccacgtgacg |
| 5101 | ctcatgaggt cccagcgcaa ctacatggtg cagacggagg accagtacag cttcatccac |
| 5161 | gaggccctgc tggaggccgt gggctgtggc aacacagaag tgcccgcacg cagcctctat |
| 5221 | gcctacatcc agaagctggc ccaggtggag cctggcgaac acgtcactgg catggaactc |
| 5281 | gagttcaagc ggctggctaa ctccaaggcc cacacgtcac gcttcatcag tgccaatctg |
| 5341 | ccttgtaaca agttcaagaa ccgcctggtg aacatcatgc cctatgagag cacacgggtc |
| 5401 | tgtctgcaac ccatccgggg tgtggagggc tctgactaca tcaacgccag cttcattgat |
| 5461 | ggctacaggc agcagaaggc ctacatcgcg acacaggggc cgctggcgga gaccacggaa |
| 5521 | gacttctggc gcatgctgtg ggagaacaat tcgacgatcg tggtgatgct gaccaagctg |
| 5581 | cgggagatgg gccgggagaa gtgtcaccag tactggccgg ccgagcgctc tgcccgctac |
| 5641 | cagtactttg tggtagatcc gatggcagaa tacaacatgc ctcagtatat cctgcgagag |
| 5701 | ttcaaggtca cagatgcccg ggatggccag tcccggactg tccggcagtt ccagttcaca |
| 5761 | gactggccgg aacagggtgt gccaaagtcg ggggagggct tcatcgactt cattggccaa |
| 5821 | gtgcataaga ctaaggagca gtttggccag gacggcccca tctctgtcca ctgcagtgcc |
| 5881 | ggcgtgggca ggacgggcgt cttcatcacg cttagcatcg tgctggagcg gatgcggtat |
| 5941 | gaaggcgtgg tggacatctt tcagacggtg aagatgctac gaacccagcg gccggccatg |
| 6001 | gtgcagacag aggatgagta ccagttctgt taccaggcgg cactggagta cctcggaagc |
| 6061 | tttgaccact atgcaaccta aagccatggt tccccccagg cccgacacca ctggccccgg |
| 6121 | atgcctctgc ccctcccggg cggacctcct gaggcctgga cccccagtgg gcagggcagg |
| 6181 | aggtggcagc ggcagcagct gtgtttctgc accatttccg aggacgacgc agcccctcga |
| 6241 | gcccccccac cggccccggc cgccccagcg acctccctgg caccggccgc cgccttcaaa |
| 6301 | tacttggcac attcctcctt tccttccaat tccaaaacca gattccgggg tggggggtgg |
| 6361 | ggggatggtg agcaaatagg agtgctcccc agaaccagag gagggtgggg cacagaccat |
| 6421 | agacggaccc ctcgtcctcc cccagcggtg gtagggggac ccggggggct cctccccgct |
| 6481 | ctgcagcctg gggacactgg gctgggacca gaatccagct ttcttttaaa actctcagtg |
| 6541 | taactgtatc ccgtgacatt tcattttttt taaatagtgt attttttttt ccattttttt |
| 6601 | ttttaagaga aacaaacaaa agactcgcca gtcaatgact ttcaaagaga actaactttg |
| 6661 | gcttattcat attctgttca aagacagtct attttttcac tgtagaaagc gtccttgtgt |
| 6721 | gatagttacg ttcgcaaacg cgcacgccag gcccatggct gtaccttggc tttttttttt |
| 6781 | tttttttttt ttttaatttt tcctaccatc agaaagtgtg ctttgctcac agaagaatgg |
| 6841 | gatgtccttt tttctttctt ggcttttttt ttcccccttt ttgtttcatt tttataaatt |
| 6901 | aaattttcag acatatcaaa tacagttctg agggtaaggt catgggggag ctcggaccca |
| 6961 | gtggcgttgg gtgcggttga gggggacgct gctgtaagag gagagagatg acagtggtcc |
| 7021 | tcctctgaga gcctgagctg tctccccgtc tcccgccccc aaggagacag agaggatcct |
| 7081 | acttcttcgg ggacagtggc tgtatggctg tgctgcccca catcagggac cctttccccc |
| 7141 | tgggactgtg gggcagtttg ggagcaaaac cagaaggaca ggcccccctc tacccgccta |
| 7201 | ccctgagcaa gcgagttgtt cctctttgta caagggcagg tctgcggtta ctttcaacac |
| 7261 | tgtttattcc agcggaagca gccgggtggt tttcccaccc ccgtgtatgt agatatatcg |
| 7321 | actttgtatt aaaggaagat cgtctga |

Murine amino acid sequence:

(SEQ ID NO: 7)
MAPTWSPSVVSVVGPVGLFLVLLARGCLAEEPPRFIREPKDQIGVSGGVASFVCQATGDPKPRVTWNKKG
KKVNSQRFETIDFDESSGAVLRIQPLRTPRDENVYECVAQNSVGEITIHAKLTVLREDQLPPGFPNIDMG
PQLKVVERTRTATMLCAASGNPDPEITWFKDFLPVDPSASNGRIKQLRSGALQIESSEETDQGKYECVAT
NSAGVRYSSPANLYVRVRRVAPRFSILPMSHEIMPGGNVNITCVAVGSPMPYVKWMQGAEDLTPEDDMPV
GRNVLELTDVKDSANYTCVAMSSLGVIEAVAQITVKSLPKAPGTPVVTENTATSITVTWDSGNPDPVSYY
VIEYKSKSQDGPYQIKEDITTTRYSIGGLSPNSEYEIWVSAVNSIGQGPPSESVVTRTGEQAPASAPRNV
QARMLSATTMIVQWEEPVEPNGLIRGYRVYYTMEPEHPVGNWQKHNVDDSLLTTVGSLLEDETYTVRVLA
FTSVGDGPLSDPIQVKTQQGVPGQPMNLRAEAKSETSIGLSWSAPRQESVIKYELLFREGDRGREVGRTF
DPTTAFVVEDLKPNTEYAFRLAARSPQGLGAFTAVVRQRTLQAKPSAPPQDVKCTSLRSTAILVSWRPPP
PETHNGALVGYSVRYRPLGSEDPDPKEVNNIPPTTTQILLEALEKWTEYRVTAVAYTEVGPGPESSPVVV
RTDEDVPSAPPRKVEAEALNATAIRVLWRSPTPGRQHGQIRGYQVHYVRMEGAEARGPPRIKDIMLADAQ
EMVITNLQPETAYSITVAAYTMKGDGARSKPKVVVTKGAVLGRPTLSVQQTPEGSLLARWEPPADAAEDP
VLGYRLQFGREDAAPATLELAAWERRFAAPAHKGATYVFRLAARGRAGLGEEAAAALSIPEDAPRGFPQI
LGAAGNVSAGSVLLRWLPPVPAERNGAIIKYTVSVREAGAPGPATETELAAAAQPGAETALTLRGLRPET
AYELRVRAHTRRGPGPFSPPLRYRLARDPVSPKNFKVKMIMKTSVLLSWEFPDNYNSPTPYKIQYNGLTL
DVDGRTTKKLITHLKPHTFYNFVLTNRGSSLGGLQQTVTARTAFNMLSGKPSVAPKPDNDGFIVVYLPDG
QSPVTVQNYFIVMVPLRKSRGGQFPVLLGSPEDMDLEELIQDISRLQRRSLRHSRQLEVPRPYIAARFSI
LPAVFHPGNQKQYGGFDNRGLEPGHRYVLFVLAVLQKNEPTFAASPFSDPFQLDNPDPQPIVDGEEGLIW
VIGPVLAVVFIICIVIAILLYKNKPDSKRKDSEPRTKCLLNNADLAPHHPKDPVEMRRINFQTPGMLSHP
PIPITDMAEHMERLKANDSLKLSQEYESIDPGQQFTWEHSNLEANKPKNRYANVIAYDHSRVILQPLEGI
MGSDYINANYVDGYRRQNAYIATQGPLPETFGDFWRMVWEQRSATVVMMTRLEEKSRIKCDQYWPNRGTE
TYGFIQVTLLDTMELATFCVRTFSLHKNGSSEKREVRHFQFTAWPDHGVPEYPTPFLAFLRRVKTCNPPD
AGPIVVHCSAGVGRTGCFIVIDAMLERIKTEKTVDVYGHVTLMRSQRNYMVQTEDQYGFIHEALLEAVGC
GNTEVPARSLYTYIQKLAQVEPGEHVTGMELEFKRLASSKAHTSRFITASLPCNKFKNRLVNILPYESSR
VCLQPIRGVEGSDYINASFIDGYRQQKAYIATQGPLAETTEDFWRALWENNSTIVVMLTKLREMGREKCH
QYWPAERSARYQYFVVDPMAEYNMPQYILREFKVTDARDGQSRTVRQFQFTDWPEQGAPKSGEGFIDFIG
QVHKTKEQFGQDGPISVHCSAGVGRTGVFITLSIVLERMRYEGVVDIFQTVKVLRTQRPAMVQTEDEYQF
CFQAALEYLGSFDHYAT

Murine nucleotide sequence:

| (SEQ ID NO: 8) | |
|---|---|
| 1 | aggggtgacg tcaccggctg ggggcgcgcg agccgcagtg gggttttgcc ccgcccgcca |
| 61 | ggcagctcgg gccgcgcgca cacgcggagc cgccggagcc cgggccgacc cggtgccggg |
| 121 | agcagcatgc ggagcccgca gacgctgccc ctctggacac ctcagcctga ggcctctccg |
| 181 | tgagtcacgg gggtaccatc ccccaccagg gcagaggctg gaggccactg ccaagcatgg |
| 241 | cgcccacctg gagtcccagc gtggtgtctg tggtgggtcc tgtggggctc ttcctcgtac |
| 301 | tgctggccag aggatgcttg gctgaagaac cacccaggtt tatcagagag cccaaggatc |
| 361 | agattggagt gtcgggaggc gtggcctcct tcgtgtgcca ggccacgggt gatcctaagc |
| 421 | cacgggtgac ctggaacaag aagggcaaga aagtgaactc acagcgcttc gagaccattg |
| 481 | actttgacga gagctctggg gcggtcctga ggatccagcc acttcggacg cctcgggatg |
| 541 | agaacgtgta cgagtgtgtg gcccagaact cggtgggcga aatcacaatt catgcaaagc |
| 601 | tcaccgtcct tcgagaggac cagctgcctc ctggcttccc caacattgac atgggccccc |
| 661 | agttgaaggt tgtagagcgc acacgcacag ccaccatgct ctgtgctgcc agcgggaacc |
| 721 | cggaccctga gatcacctgg tttaaggact tcctgcctgt ggaccccagt gccagcaacg |
| 781 | ggcggatcaa gcagcttcga tcaggtgccc tgcagattga gagcagcgag gagacagacc |
| 841 | agggcaagta cgagtgtgtg gccaccaaca gcgctggggt gcgctactca tcacctgcca |
| 901 | acctctacgt gcgagtccgc cgtgtggccc cacgcttctc catcctgccc atgagccacg |
| 961 | agatcatgcc cggtgggaat gtgaatatca cttgtgtggc cgtgggctca cccatgccct |
| 1021 | acgtgaaatg gatgcagggg gccgaggacc tgacgcctga ggatgacatg cccgtgggtc |
| 1081 | ggaatgttct agaactcacg gatgtcaagg actcagctaa ctacacttgt gtggccatgt |
| 1141 | ccagcctggg tgtgatcgag gccgtggccc agatcactgt aaaatctctc cccaaagccc |
| 1201 | ctgggactcc tgtggtgacg gagaacactg ccaccagtat cactgtcaca tgggactcgg |
| 1261 | gcaaccctga ccccgtgtcc tactacgtaa ttgagtataa gtccaaaagc caggatgggc |
| 1321 | cgtatcagat caaagaagac atcaccacca cgcgctacag catcggaggc ctgagcccca |
| 1381 | attctgagta tgagatctgg gtgtcagctg tcaactccat tggccagggc cctcccagtg |
| 1441 | aatcggtggt gacccgcaca ggtgagcagg caccagccag cgctcccagg aatgttcagg |
| 1501 | cccgcatgct cagcgccacc accatgatcg tgcagtggga ggagcctgtg gagcccaatg |
| 1561 | gcctgatccg tggctaccgt gtctactata ccatggagcc ggaacaccca gtgggcaact |
| 1621 | ggcagaaaca caatgtggac gacagtctcc tgaccactgt gggcagcctg ctggaagacg |
| 1681 | agacctacac cgtgcgcgtg ctcgccttca cgtcggtggg cgacggacca ctgtcagacc |
| 1741 | ccatccaggt caagacccag cagggagttc ctggccagcc catgaacttg cgggctgagg |
| 1801 | ccaagtcaga gaccagcatt gggctctcgt ggagtgcacc acgacaggag agtgtcatta |
| 1861 | agtatgaact gctcttccgg gagggcgacc gaggccgaga ggtggggcga accttcgacc |
| 1921 | caaccacagc ctttgtggtg gaggacctca agcccaatac ggagtatgcg ttccggctgg |
| 1981 | cggcgcgctc gccgcagggc ctgggcgcct tcaccgcggt cgtgcgccag cgcacgctgc |
| 2041 | aggccaaacc gtcagccccc cctcaagacg ttaagtgcac cagcttgcgc tccacggcca |
| 2101 | tattggtaag ttggcgcccg ccaccgccag aaactcacaa cggggccctc gtgggctaca |
| 2161 | gcgtccgcta ccgaccgctg ggctcagagg acccggaccc caaggaggtg aacaacatac |
| 2221 | ccccgaccac cactcagatc cttctggaag ctttggagaa atggacggag taccgtgtca |
| 2281 | ccgccgtggc ttacacagag gtgggaccag ggcccgagag ctcgcccgtg gtcgtccgca |
| 2341 | ccgatgagga cgtgcccagc gcgcccccgc ggaaggtgga ggcggaggcg ctcaacgcca |
| 2401 | cagccatccg agtgctgtgg cgctcgccca cgcccggccg gcagcacggg cagatccgcg |
| 2461 | gctaccaggt ccactatgtg cgcatggagg gtgccgaggc ccgcgggcca ccgcgcatca |
| 2521 | aggacatcat gctggcggat gcccaggaaa tggtgataac gaacctccag cctgagactg |
| 2581 | cttactctat cacagtagcc gcgtatacca tgaaaggcga tggcgctcgc agcaaaccga |
| 2641 | aggtggtggt gaccaaggga gcagtgctgg gccgccccac cctgtcggtg cagcagaccc |
| 2701 | ccgagggcag cctgctggcg cgctgggagc cccccgcgga cgcggccgag gacccggtgc |
| 2761 | ttggctaccg cctgcagttt gggcgcgaag acgcggcccc ggccacgttg gagctggctg |
| 2821 | cgtgggagcg gcggttcgcg gcgcctgcac acaagggcgc cacctatgtg ttccggctgg |
| 2881 | cagcgcgggg ccgcgcgggg ttgggcgagg aggccgcggc agcgctgagc atccccgagg |
| 2941 | acgctccgcg cggcttcccg cagatcttgg gcgccgcggg caacgtgtcc gcgggctccg |
| 3001 | tgctactgcg ctggctgcca cccgtgcccg ccgagcgcaa cggcgccatc atcaagtaca |
| 3061 | cggtgtccgt gcgggaggcc ggcgcccctg ggcccgcgac cgagacggag ctggcggcgg |
| 3121 | ccgcccagcc gggggccgag acagcgctca cgctgcgagg gctgcggccg gagacggcct |
| 3181 | acgagttacg cgtgcgcgca cacacgcgtc gcggcccggg ccccttctca cccccgctgc |
| 3241 | gctacaggct cgcgcgggac ccagtctccc caaagaactt caaggtgaag atgatcatga |
| 3301 | agacttcagt gctgctgagc tgggagttcc ccgacaacta taactcaccc acaccctaca |
| 3361 | agattcagta caatgggctc accctggatg tggacggccg cacgaccaag aagctgatca |
| 3421 | cacacctcaa gccacacacc ttctataatt tcgtgctcac caaccgtggc agcagcctgg |
| 3481 | ggggcctgca gcagacggtc actgccagga ccgcctttaa catgctcagt ggcaagccta |
| 3541 | gcgtcgcccc gaagcccgac aatgacggtt tcatcgtggt ctacctgcct gatggccaga |
| 3601 | gtcctgtgac cgtgcagaac tacttcattg tgatggtccc acttcggaag tctcgaggtg |
| 3661 | gccagttccc tgtcctacta ggtagtccag aggacatgga tctggaggag ctcatccagg |
| 3721 | acatctcccg gctgcagagg cgcagcctgc gccactccag acagctggag gtgcctcggc |
| 3781 | cctacatcgc cgctcgattc tccatcctgc cagctgtctt ccatcctggg aaccagaagc |
| 3841 | aatatggtgg ctttgacaac aggggcttgg agccaggcca ccgctatgtc ctctttgtgc |
| 3901 | ttgctgtgtt gcagaagaat gagcctacat ttgcagccag tcccttctca gaccccttcc |
| 3961 | agctggacaa cccggaccct cagcccattg tggacggcga ggagggcctc atctgggtga |
| 4021 | ttgggcctgt gctggccgtg gtcttcatca tctgcatcgt gattgccatc ctgctgtaca |
| 4081 | agaacaaacc tgacagcaaa cgcaaggact cagagccccg caccaaatgc ttactgaaca |
| 4141 | atgccgacct tgccccccat caccccaagg accctgtgga aatgcgacgc atcaacttcc |
| 4201 | agacaccagg tatgctcagc cacccaccca tccccatcac agacatggcg gagcacatgg |
| 4261 | agagactcaa agccaacgac agcctgaagc tctcccagga gtacgagtcc attgaccccg |
| 4321 | ggcagcaatt cacgtgggaa cattcgaacc tggaggccaa caagcccaag aaccgctatg |
| 4381 | ccaacgtcat cgcctatgac cactcacgag tcatcctgca gcccctagaa ggcatcatgg |
| 4441 | gtagtgatta catcaatgcc aactatgtgg acggctaccg gcggcagaat gcatacattg |
| 4501 | ccacgcaggg gcccctgcct gagacctttg gggacttctg gcggatggtg tgggagcagc |
| 4561 | gatcggccac tgtggtcatg atgacgcgac tggaggagaa atcacggatc aaatgtgacc |
| 4621 | aatactggcc taaccgaggc accgagacat acggcttcat ccaggtcacc ctactagata |
| 4681 | ccatggagct ggctaccttc tgcgtcagga ctttttctct acacaagaat ggctctagcg |
| 4741 | agaagcgtga ggtgcgacat ttccagttca cggcatggcc cgaccacggg gtacctgagt |
| 4801 | accccacgcc cttcctggca ttcctgcgaa gagtcaagac ctgcaacccg cctgatgctg |
| 4861 | gccccattgt ggtccactgc agcgcgggtg tggggcgcac tggctgcttc atcgtaattg |
| 4921 | acgccatgct agagcgcatc aagacagaga agaccgtgga tgtgtatgga catgtgacac |
| 4981 | tcatgcggtc gcagcgcaac tacatggtgc agacagagga tcagtatggc ttcatccacg |
| 5041 | aggcgctgct ggaggctgtg ggctgcggca ataccgaggt ccctgctcgc agcctctaca |
| 5101 | cctacatcca gaagctggcc caggtggagc ctggcgagca cgtcacgggc atggagcttg |
| 5161 | agttcaagag gctcgccagt tccaaggcac acacttcgcg cttcatcacc gccagcctgc |
| 5221 | cttgcaacaa gtttaagaac cgactggtga acatcctgcc gtacgagagc tcgcgtgtct |
| 5281 | gcctgcagcc catccgcggt gtggagggct ctgactacat caatgccagc tttatcgacg |
| 5341 | gctatagaca gcagaaagcc tacattgcaa cacaggggcc actggcagag accacagagg |
| 5401 | acttctggcg agctctgtgg gagaacaact ctactattgt cgtaatgctc accaagctcc |
| 5461 | gagaaatggg ccgggaaaag tgccaccagt actggccagc cgagcgctct gcccgctacc |
| 5521 | agtactttgt ggttgacccg atggcagagt ataacatgcc acagtacatt ctgcgtgagt |
| 5581 | ttaaggtcac agatgcccgg gatggccagt cccggaccgt ccgacagttc cagttcacgg |
| 5641 | actggccaga gcagggtgca cccaagtcag gggaaggctt cattgacttc atcggccaag |
| 5701 | tgcataagac caaggagcag tttggccagg acggacccat ctcagtgcac tgcagcgccg |
| 5761 | gagtgggcag gaccggagtg ttcatcaccc tgagcatcgt gcttgagcgg atgcgctacg |
| 5821 | agggcgtggt ggacattttc cagacagtga aggtgcttcg gacccagagg cctgccatgg |
| 5881 | tgcagacaga ggacgagtac cagttctgct tccaggcggc tttggaatac ctgggcagtt |
| 5941 | ttgatcatta tgcaacataa gccatgggcc ccgcccaaca cctcagccct gcgccaagtg |
| 6001 | ccctggatgt gagcctaggc ccgccgctgg gcaggatgcg gcccagggag acctcctctt |
| 6061 | cgcggagaca ggcgctgcct tcctcattcc cttctgattc caaaacgagg ttccagggtg |
| 6121 | gggggttggg gtggagagag aaggagccac tgctccccag gctggggtca cacagggacc |
| 6181 | gacctctgct tccgcactcc cctgcctgcc ttttggcaac attttttttc ttattttttt |
| 6241 | ttaatagtgt atattttttt tctttttctt tttttctttt ttttttttaa gaaaaaaaca |
| 6301 | aaatcgtgcc ggtcaaaact ttgaaaaaga aacaagatca ctgtttgtgc ctctgtggga |
| 6361 | ggcctatttt ttcatagtta gtgtgccgtg tggcggctat gtgcggccac ttcgacggct |
| 6421 | tctgtgtgtg catctttccc acatgcccga cactgccccc atccccatgt gaatggtgcg |
| 6481 | cttagttttt atttttaacc tttttacttt ttttttaatc aatcttcaga catatcagat |
| 6541 | atggagggtg aggcgctggg ggcactcggg ccagactaca gggacatggc caccaaggac |
| 6601 | acagtggctg gccttgctgc tccagtccct ggcacaccag ggagggtcct cgtctactca |
| 6661 | tgacctctgt gccccgcatg gaggacctgg gactacggga cacttggggg atatccaacc |
| 6721 | ccctggagca actgaggtct ctctttgtag gagagtgggt cagtactcgt ccccgctgtt |
| 6781 | ttttgggcag aagcagcagg tgacgcccct gtatgtagat aaaccaactt tgtattaaag |
| 6841 | aaagattcgt ccgacctaga aaaaaaaaaa aaaa |

Additional information and sequences regarding PTPσ and its functional domains (e.g., first immunoglobulin-like domain) are available in Aricescu et al., 22 Mol. Cell. Biol. 1881 (2002), as well as the following GENBANK Accession Numbers: NM 130855, NM 130854, NM 130853, NM 002850 for human; and NM 011218 for mouse. For example, a purified PTPσ fragment that includes a first immunoglobulin-like domain comprises residues 47-109 or 33-123 of the amino acid sequence of NM 002850.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The present invention may be as defined in any one of the following numbered paragraphs.
1. A method of promoting outgrowth of a neuron, comprising contacting the neuron with an agent that inhibits the interaction of chondroitin sulfate proteoglycan (CSPG) to protein tyrosine phosphatase sigma (PTPσ).
2. The method of paragraph 1, wherein the neuron is in vivo.
3. The method of paragraph 1, wherein the neuron is in vitro.
4. A method of promoting neural regeneration in the central nervous system (CNS) of a subject in need thereof, comprising contacting a neuron of the subject with an agent that inhibits the interaction of chondroitin sulfate proteoglycan (CSPG) to protein tyrosine phosphatase sigma (PTPσ).
5. The method of paragraphs 1-4, wherein the agent inhibits binding of CSPG to PTPσ.
6. The method of paragraphs 1, 2, 4 and 5, wherein the neuron is located at a site of injured or diseased tissue.
7. The method of paragraphs 1, 2, 4, and 5, wherein the neuron is located at a site of spinal cord injury.
8. The method of paragraphs 1-7, wherein the agent binds to the first immunoglobulin-like domain of PTPσ.
9. The method of paragraphs 1-7, wherein the agent comprises a soluble PTPσ polypeptide or portion or fragment thereof.
10. The method of paragraphs 1-7, wherein the agent comprises a soluble PTPσ ectodomain or a portion or fragment thereof.
11. The method of paragraphs 1-7, wherein the agent comprises a molecule that binds to the CSPG binding site of PTPσ, or that binds to the PTPσ binding site of CSPG.
12. The method of paragraphs 1-7, wherein the agent is a PTPσ-specific antibody or portion or fragment thereof, the epitope binding specificity of which comprises the first immunoglobulin-like domain of PTPσ.
13. The method of paragraphs 1-12, further comprising contacting the neuron with a second agent, wherein the second agent inhibits a myelin inhibitor of neural regeneration.
14. A method of identifying a compound that reduces CSPG inhibition of neural regeneration, comprising providing a polypeptide comprising first immunoglobulin-like domain of PTPσ, contacting the polypeptide with a candidate compound, wherein binding of the candidate compound to the polypeptide indicates that the candidate compound reduces binding of CSPG to PTPσ, thereby reducing CSPG inhibition of neural regeneration.
15. A method of identifying a site of neural injury by administering to a subject a detectably-labeled PTPσ or a fragment thereof which binds to CSPG, wherein localization of the PTPσ or fragment thereof at an anatomical location indicates that the location is a site of neural injury.
16. The method of paragraph 15, wherein the fragment comprises the first immunoglobulin-like domain of PTPσ.
17. A method of inhibiting the interaction of chondroitin sulfate proteoglycan (CSPG) to transmembrane Protein Tyrosine Phosphatase sigma (PTPσ) in a cell, comprising contacting the cell with an agent that inhibits CSPG binding to PTPσ.
18. The method of paragraph 17, wherein the cell is a neuron.
19. The method of paragraph 18 wherein the neuron is a central nervous system neuron.
20. The method of paragraph 18 wherein the neuron is a peripheral nervous system neuron.
21. The method of paragraph 17, wherein the cell is a non-neuronal cell that provides support to neural cells.
22. The method of paragraphs 17-21, wherein the agent is isolated/purified CSPG or a portion or fragment thereof.
23. The method of paragraph 22, wherein the fragment of CSPG is chondroitin sulfate or a portion thereof.
24. The method of paragraphs 17-21, wherein the agent is isolated/purified antibody which specifically binds an epitope on CSPG.
25. The method of paragraph 24, wherein the epitope is on chondroitin sulfate.
26. The method of paragraphs 17-21, wherein the agent is an isolated/purified antibody or fragment thereof which specifically binds an epitope on an immunoglobulin-like domain of PTPσ.
27. The method of paragraph 26, wherein the epitope is on the first immunoglobulin-like domain of PTPσ.
28. The method of paragraphs 17-21, wherein the agent is an isolated/purified soluble PTPσ polypeptide.
29. The method of paragraphs 17-21, wherein the agent is a purified/isolated soluble PTPσ ectodomain.
30. The method of paragraphs 17-21, wherein the agent is a small molecule.

The invention is further illustrated by the following examples, which should not be construed as further limiting.

### EXAMPLES

### Example 1

### PTPσ Interacts with the CS Chains of CSPGs

The Ncn-AP fusion protein construct was generated by PCR amplification of nucleotide 1-2889 of a full-length mouse neurocan clone (Accession: BC065118, IMAGE:6853253) and subcloning the neurocan fragment into the NheI and HindIII sites of the APtag5 vector. See Flanagan et al., 327 Meth. Enzymol. 19 (2000). The resulting fusion protein includes neurocan amino acids 1-963 (N-terminal Ig domain, tandem link domains and central chondroitin sulfate attachment domain) fused to placental alkaline phosphatase. PTPσ-Fc and PTPσ-AP fusion protein constructs were generated by PCR amplification of nucleotide 1-2538 of a full-length mouse PTPσ clone (Accession BC052462, IMAGE 6834684), encoding the short isoform of PTPσ, which differs from the long isoform by alternative splicing to give 4 or 8 fibronectin domains. Chagnon et al., 82 Biochem. Cell Bio. 664 (2004); Sajnani-Perez et al., 22 Mol. Cell. Neurosci. 37 (2003). The PTP fragment was subcloned into the NheI and HindIII sites of the pSectagIg and APtag5 vectors to give Fc and AP fusions respectively. To generate the PTPσ ΔLys constructs, lysine residues K68, K69, K71 and K72 in the N-terminal Ig domain (Aricescu et al., 22 Mol. Cell. Biol. 1881 (2002)) were simultaneously changed to alanines using PCR.

Fusion proteins were produced in transiently transfected 293T cells. For purification, proteins were produced in Opti-MEM plus ITS-A (Invitrogen), and purified with protein A-Sepharose beads (4 Fast Flow, Amersham). The AP activity of the fusion proteins was determined by measuring substrate turnover on a microplate reader as described elsewhere. Flanagan et al., 2005; Flanagan & Cheng, 327 Meth. Enzymol. 198 (2000).

PTPσ^{-/-} mice (Silver & Miller, 2004) were kindly provided by Dr. Michel Tremblay. Mouse C8-D1A cerebellar astrocytes were from the ATCC and were maintained in DMEM containing 10% bovine calf serum (Invitrogen).

Regarding chondroitinase digestion, for cell free binding assays, chondroitinase ABC (Sigma) was reconstituted to 0.5 mg/ml in 0.01% BSA and added at a final concentration of 0.05 mg/ml (5 units/ml) to 100 μl of 30 nM Ncn-AP. For treatment of astrocytes, cultures were gently rinsed 3 times in culture dishes with warm DMEM, and incubated at 37° C. with chondroitinase ABC (1 unit/ml) in enzyme buffer (100 mM Tris, pH=8.0, 100 mM sodium acetate and 0.02% bovine serum albumin). Cells were then rinsed with HBAH before binding assays with AP fusion proteins. To test the effect on DRG neuron culture, for each culture well, chondroitinase ABC (0.2 unit) was mixed with or without CSPGs (25 μg; Millipore) in enzyme buffer in a total volume of 100 μl. The mixtures were incubated at 37° C. for 3 hours and then added to the culture medium for outgrowth assays. Spinal cord cryosections were rinsed with Hanks Buffered Saline Solution (HBSS) and incubated at 37° C. for 3 hours with chondroitinase ABC (1 unit/ml) in enzyme buffer, then were rinsed with HBSS before fusion protein binding assays.

In cell free binding assays to test fusion proteins labeled with an AP tag for binding to fusion proteins labeled with an Fc tag, binding assays were performed using 96-well Reactibind Protein A-coated plates (Pierce) as described. Rhodes & Fawcett, 2004. Wells were washed with HBSS containing 20 mM Hepes pH 7.0 and incubated with PTPσ-Fc or control Fc conditioned media (4 μg/ml) for 2 hr. Wells were blocked in HBAH (HBSS with 0.5 mg/ml BSA and 0.1% NaN₃) for 2 hr and incubated with Ncn-AP or control AP for 1.5 hr. Unless otherwise stated, all AP fusion proteins were normalized for equal AP activity. Wells were washed five times with HBAH and assayed for bound AP activity as described elsewhere. Case & Tessier-Lavigne, 2005; Yiu & He, 2006. For experiments involving treatment with chondroitinase ABC (Sigma), chondoitinase-treated or mock-treated Ncn-AP were incubated at 37° C. for 2 hr, added to PTPσ-Fc coated Reactibind Protein A plate wells and assayed for binding.

For experiments to test binding of aggrecan or CS to PTPσ-Fc, purified aggrecan (bovine; Sigma) was dissolved at 2 mg/ml in HBSS, and was biotinylated by mixing 20 μl aggrecan with 12 μl of 10 mM EZ-Link Sulfo-NHS-LC-LC-Biotin (Pierce) in PBS in a total volume of 200 μl, and incubating at 37° C. for 3 hr. Purified chondroitin sulfate (Sigma #C4384) was dissolved at 2 mg/ml in water, and was similarly biotinylated by mixing 200 μl CS with 28 μl of 10 mM Sulfo-NHS-LC-LC-Biotin. Unincorporated biotin was removed using a Zeba Desalt Spin Column (Pierce). 100 μl of PTPσ-Fc or control Fc conditioned media (4 μg/ml) was added to Reactibind Protein A-coated microtitre plate wells (Pierce) and incubated at room temperature for 1 hr. Following five HBAH washes, biotinylated aggrecan or CS was added at the indicated concentrations and the plates incubated at room temperature for 1 hr. Wells were washed ten times with HBAH and incubated with alkaline phosphatase-conjugated streptavidin (Pierce) at room temperature for 1 hr. Wells were then washed ten times with HBAH and assayed for bound AP activity as described elsewhere. Flanagan et al., 2000; Flanagan & Cheng, 2000.

For PTPσ fusion protein binding to cells or tissue, astrocyte cultures were rinsed with PBS and pre-incubated with blocking buffer (10% normal goat serum and 0.1% NaN3 PBS) at room temperature for 1 hr without or with 20 μg/ml anti-CS antibody (CS56, Sigma), or a control antibody matched for the IgM isotype (anti-His, Santa Cruz). Cells were then rinsed with HBAH and incubated for 1.5 hr with 20 nM PTPσ-AP or AP control. Cell cultures were rinsed five times with HBAH, lysed, and bound AP activity was measured in microplates with a spectrophotometer as described. Flanagan et al., 2000; Flanagan & Cheng, 2000.

For binding experiments with lesioned spinal cord cryosections, mice were anesthetized with mixture of ketamine (100 mg/kg) and xylazine (10 mg/kg), and a T8 laminectomy was performed. To produce a dorsal hemisection injury, the dorsal spinal cord was first cut with a pair of microscissors and then a fine microknife was drawn bilaterally across the dorsal aspect of the spinal cord. After hemostasis was achieved, the muscle layers and the skin were sutured. Seven days post-lesion, mice were perfused with 4% paraformaldehyde and post-fixed over-night, then the spinal cords were cryo-proteced in 30% sucrose, and 20 μm sagittal cryosections were cut. Spinal cord cryosections were then probed with AP fusion proteins as previously described (Flanagan et al., 2000; Flanagan & Cheng, 2000.) or by immunofluorescence as described herein.

For Immunolocalization studies, astrocytes were cultured on coverslips and fixed with 4% paraformaldehyde at room temperature for 15 min. Blocking was with 10% normal goat serum in PBS without detergent, without or with addition of CS56 antibody (20 μg/ml; Sigma). Cells were then incubated at room temperature with purified PTPσ-Fc (20 μg/ml) in blocking buffer for 1.5 hr and goat anti-human IgG conjugated with Alexa Fluor 488 (1 μg/ml; Molecular Probes) for an additional 1.5 hr. After brief treatment with DAPI nuclear counterstain and three rinses with PBS, samples were mounted with Fluoromount (SouthernBiotech) before imaging.

Cultured DRG neurons were fixed with 4% paraformaldehyde at room temperature for 30 min, rinsed with PBS and pre-incubated for 1 hr in blocking buffer (10% normal donkey serum and 0.4% Triton-X100 in PBS). Cells were then incubated in blocking buffer with anti-GAP43 antibody (2.5 μg/ml; Novus) and subsequently donkey anti-rabbit antibody conjugated with Alexa Fluor 488 (1 μg/ml; Molecular Probes), each overnight at 4° C. After brief treatment with DAPI nuclear counterstain and three rinses with PBS, samples were mounted with Fluoromount (SouthernBiotech) before imaging.

Spinal cord cryosections were rinsed with HBSS and blocked for 1 hour in 10% normal goat serum in PBS without detergent. Primary antibodies (anti-neurocan or CS56, Sigma; each 20 μg/ml) were mixed with either Fc or PTPσ-Fc (each 20 μg/ml) in blocking buffer and incubated with cryosections for 1.5 hr at room temperature. Sections were gently rinsed and incubated with secondary antibodies in blocking buffer for 1.5 hr at room temperature (goat anti-mouse antibody conjugated with Alexa Fluor® 594 or goat anti-human IgG antibody conjugated with Alexa Fluor® 488, Molecular Probes, each 1 μg/ml). Sections were then rinsed three times in PBS and mounted with Fluoromount (SouthernBiotech) before imaging.

For immunoprecipitation and western blots, astrocyte cell lysates were grown in 10 cm culture dishes, rinsed with cold PBS and lysed for 30 min on ice in buffer containing 100 mM Tris, pH 8.0, 1 mM EDTA, 1% NP40, 1% Octyl glucoside and protease inhibitors (Roche). Lysates were cleared by microcentrifugation. 1 mg of total protein was mixed with 10 μg of Fc fusion proteins and 30 μl protein A sepharose beads (50% slurry; Amersham) in lysis buffer in a total volume of 1 ml, and gently rotated overnight at 4° C. Beads were washed five times with cold lysis buffer and incubated with sample loading buffer at room temperature for 1 hr before electrophoresis. Western blot analysis was performed with anti-neurocan (0.2 μg/ml, Sigma) as described previously. Johnson et al., 49 Neuron 517 (2006).

For the DRG neurite outgrowth assay, 8-well culture slides (BD Biosciences) were briefly coated with nitrocellulose methanol solution, dried and subsequently coated with proteins at 37° C. for 2 hr. In experiments with soluble CSPGs, slides were coated with a 100 μl mixture of poly-D-lysine (200 μg/ml; Sigma) and laminin (5 μg/ml; BD Biosciences). In experiments with MAG, the slides were coated with a 100 μl mixture of poly-D-lysine (200 μg/ml), laminin (5 μg/ml) and either Fc or MAG-Fc (each 50 μg/ml; R&D). In experiments with neurocan, the slides were coated with a 100 μl mixture of poly-D-lysine (200 μg/ml), laminin (10 μg/ml) and neurocan (5 μg/ml; Millipore or US Biologicals). Coated slides were rinsed with PBS immediately before use, taking care to avoid drying. For experiments with NGF, culture slides precoated with poly-D-lysine were purchased from BD Biosciences.

DRGs from P8-12 PTPs +/+ or −/− mice were cut off from all roots. Neurons were dissociated as described (Malin et al. 2 Nat. Protoc. 152 (2007)), with modification. DRGs were rinsed in PBS with 1 mM EDTA and allowed to settle without centrifugation. Enzyme digestion was carried out sequentially with collagenase II (4 mg/ml, Worthington) and trypsin (0.25%, Gibco) each for 15 min at 37° C. DRGs were then gently rinsed with DMEM containing 10% BCS and then with culture medium (Neurobasal medium supplemented with 2% B27, 20 nm L-glutamine and 1% penicillin/streptomycin, all from Invitrogen). Dissociated cells were filtered using a cell strainer (BD Biosciences). Approximately 2000 cells suspended in 500 ml medium were seeded into each well of the culture slides.

For experiments with CSPG mixture, cells were first allowed to attach and start growing for 18 hr, then replenished with fresh medium with or without treatment reagents, and cultured for an additional 24 hr. The CSPG mixture contains large, extracellular CSPGs isolated from embryonic chicken brain; the major components are neurocan, phosphacan, versican, and aggrecan (Millipore cat#CC1 17). Unless otherwise stated, cultures contained no neurotrophins. For experiments with NGF, the culture medium was supplemented with 50 ng/ml NGF (Sigma) 30 min after seeding, then cells were analyzed 24 hr later. For experiments with MAG or neurocan, cell were grown on substrates coated with the protein of interest for 48 hr. After culturing, DRG neurons were fixed and immunostained for GAP43 positive neurons, and imaged by scanning the entire wells using a Nikon Ti inverted fluorescence microscope. The total length of neurites and total neuron number for each well was measured using MetaMorph Neurite Outgrowth software, and used to calculate average outgrowth length per neuron.

Regarding adult DRG neuron spot assay (Tom et al., 24 J. Neurosci. 6531 (2004)), glass coverslips were coated with nitrocellulose and poly-L-lysine, and spotted with a 2 μl solution of aggrecan (0.6 mg/ml; Sigma) and laminin (5 μg/ml; Invitrogen) in calcium- and magnesium-free HBSS (CMF-HBSS, Invitrogen). These spots were allowed to dry completely and covered with a laminin bath (5 μg/ml in CMF-HBSS) at 37° C. until immediately before cell plating (˜5 hr).

Dorsal root ganglion neurons were dissociated from adult (>P21) wild type or PTPσ^{-/-} mice as described previously (Horn et al., 28 J. Neurosci. 9330 (2008)), with modifications. Briefly, DRGs were removed and their central and peripheral roots were cut off. Ganglia were incubated in collagenase II (200 U/ml; Worthington) and dispase II (2.5 U/ml; Roche) in HBSS. DRGs were rinsed and triturated in CMF-HBSS three times followed by low-speed centrifugation and removal of the supernatant. Dissociated DRGs were resuspended in Neurobasal-A media supplemented with B-27, Glutamax and penicillin/streptomycin (all from Invitrogen), counted and plated at a density of 2500 cells per coverslip. After 5 days in vitro cultures were fixed in 4% paraformaldehyde in PBS for 30 min, washed three times in PBS, and incubated in blocking solution for 2 hr at room temperature. Coverslips were incubated in primary antibody overnight. Primary antibodies used were anti-CS(CS56) and anti-β-tubulin III (both 1:500; Sigma) and were followed by appropriate secondary antibodies (Molecular Probes). The average number of β-tubulin expressing neurites growing from the interior of the spot and crossing the outermost rim (visualized by CS56) were counted.

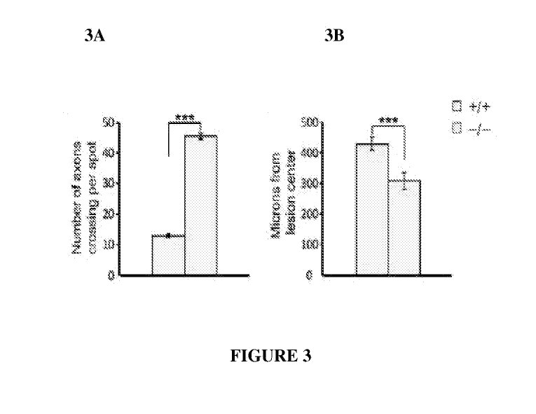

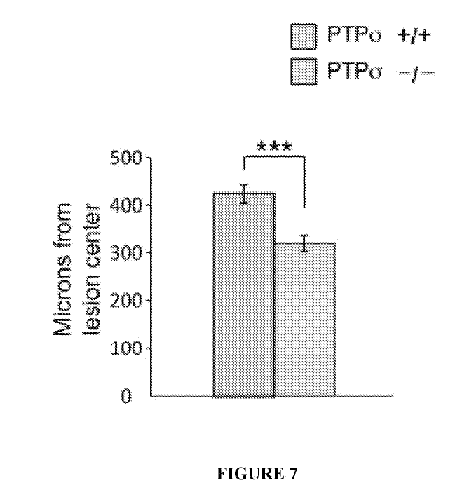

A spinal cord dorsal column crush lesion model was used for in vivo experiments to examine the effect of a lesion on sensory fibers. Adult female mice were anesthetized with inhaled isofluorane gas (2%) for all surgical procedures. All procedures were performed in a blinded fashion. A dorsal column crush spinal cord injury was performed as described previously (Horn et al., 2008), with modifications. Briefly, a T1 laminectomy was performed to expose the C8 spinal cord segment. Small holes were made in the dura 0.5 mm lateral to the midline with a 30 gauge needle and a dorsal column crush lesion was made by inserting Dumont #3 jeweler's forceps approximately 0.5 mm into the dorsal spinal cord and squeezing, holding pressure for 10 sec and repeating two additional times. The holes in the dura were covered with gel film, and the muscle layers were closed with sutures and the skin with surgical staples. The animals received Marcaine (1.0 mg/kg) subcutaneously along the incision as well as buprenorphine (0.1 mg/kg) intramuscularly. The dorsal columns were labeled unilaterally with Texas Red®-conjugated dextran 3000 MW (Invitrogen) 2 days prior to perfusion. The sciatic nerve of the right hindlimb was exposed and crushed with forceps, and a total of 1 μl of dextran was injected via a Hamilton syringe into the sciatic nerve at the crush site. Animals were killed at 14 days post spinal cord injury with an overdose of isofluorane and perfused intracardially with PBS followed by 4% paraformaldehyde. Tissue was harvested and postfixed in 4% paraformaldehyde overnight, cryoprotected in 30% sucrose, frozen in OCT mounting media and cut into 20 μm thick longitudinal sections using a cryostat. Tissue was stained with anti-GFAP antibody (Accurate Chemical & Scientific) and CS56 antibody (Sigma) and incubated with Alexa Fluor® 488 and Alexa Fluor® 647 secondary antibodies (Invitrogen), respectively. Sections were imaged on a Zeiss Axiovert 510 laser scanning confocal microscope. All measurements were made in a blinded fashion. The lesion center was determined by the characteristic GFAP staining profile. Two separate quantification paradigms were employed to determine the average position of injured fibers. First, for the quantitation shown in FIG. 3B, the distances between the endings of the five labeled axons closest to the center of the lesion were measured using Zeiss LSM 5 Image Browser software. The ventral-most sections from each animal were used in this quantification paradigm because the deepest part of the lesion tends to be most anatomically consistent between animals. Second, for the quantitation in FIG. 7, the “fiber front” was defined as the point closest to the center of the lesion at which the labeled axons formed a fascicle of closely apposed fibers approximately 30 μm wide. The distance from this “fiber front” to the center of the lesion was then measured. The measurements were taken from every section containing a fasicle of at least 30 μm in every animal using Zeiss LSM 5 Image Browser software.

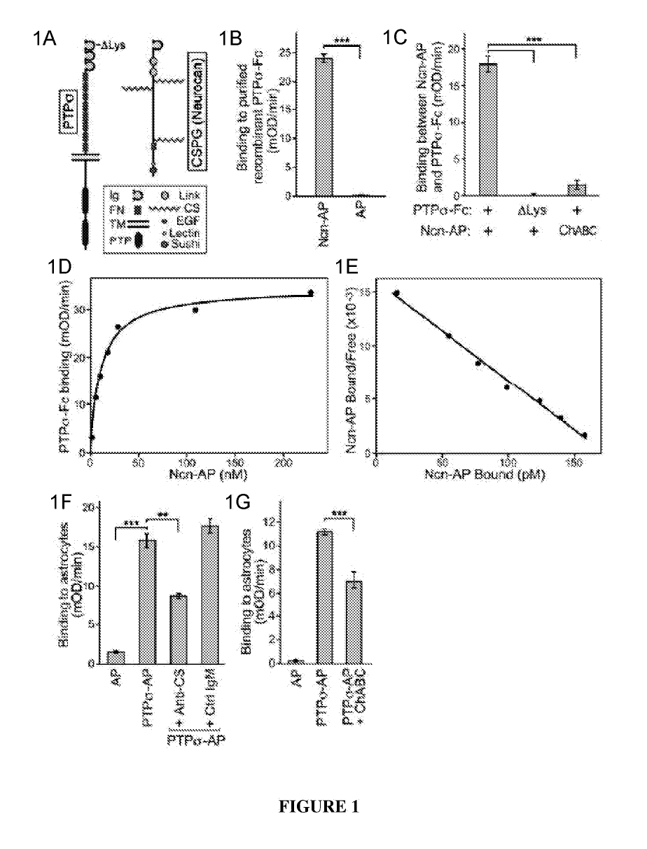

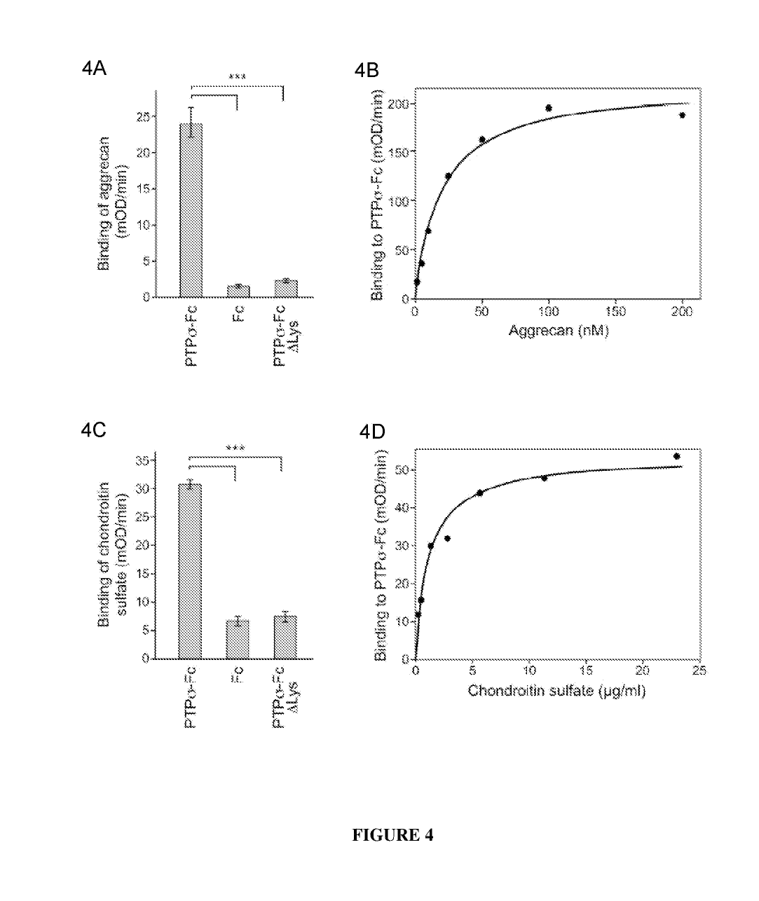

The LAR subfamily PTPs that are developmental receptors for HSPGs were investigated to determine whether they might also be receptors for CSPGs. The CSPGs and HSPGs are analogous structurally, both consisting of a core protein bearing negatively charged sulfated carbohydrate side chains. PTPσ, together with neurocan/CSPG3, a major CSPG produced by reactive astroglia following CNS injury (Asher et al., 20 J. Neurosci. 2427 (2000)) was used to test for binding (FIG. 1A). Using a cell free system, with recombinant tagged fusion proteins of the PTPσ extracellular domain (PTPσ-Fc) and neurocan (Ncn-AP) a binding interaction was indeed identified (p<0.001) (FIG. 1B). Genuine biological ligand-receptor interactions are expected to show saturability and high affinity. Adding increasing amounts of Ncn-AP to PTPσ-Fc showed that the interaction is saturable (FIG. 1D), and Scatchard analysis produced a linear plot, indicating a single binding affinity with a dissociation constant (K_{D}) of approximately 11 nM (FIG. 1E). Binding was also demonstrated with aggrecan, another astroglial CSPG, producing a K_{D} of approximately 19 nM (FIG. 4). These K_{D}s are similar to those we measured previously for the functional binding of Drosophila LAR to HSPG ligands it interacts with during development (Johnson et al., 49 Neuron 517 (2006)), and are within the typical nanomolar range for biologically significant ligand-receptor interactions. Error bars show SEM; p values were calculated by Student's unpaired t test.

The CS moiety plays an important role in CSPG mediated inhibition of neural regeneration. Bradbury et al., 2002; Houle et al., 2006; Snow et al., 1990; Gilbert et al., 2005. Experiments were carried out to test whether the CS moiety of neurocan is involved in its interaction with PTPσ. Pretreatment of the Ncn-AP fusion protein with chondroitinase ABC abolished most binding to PTPσ, confirming involvement of the CS chains (p<0.001; FIG. 1C). Some binding remained, which might be due to incomplete digestion by chondroitinase ABC, which leaves a stub of CS. Other experiments showed that PTPσ binds to isolated CS chains (FIG. 4). Although it is possible that PTPσ might also interact with the core protein of CSPGs, these experiments indicate an involvement of the CS chains.

We also investigated the binding site on PTPσ. PTPσ has a conserved positively charged region on the surface of the first immunoglobulin-like domain, and mutations of basic residues at this site impair binding of HS (Aricescu et al., 22 Mol. Cell. Bio. 1881 (2002)). Because CS, like HS, is a negatively charged carbohydrate, it seemed plausible that this site might also bind CS. A cluster of four lysine residues in this domain, K67, K68, K70 and K71, were substituted with alanines (ΔLys mutant of PTPσ; see FIG. 1A). This reduced binding to background levels (p<0.001; FIG. 1C; FIG. 4), identifying a CS interaction site on PTPσ.

Studies were carried out to ascertain whether PTPσ interacts with CSPG produced endogenously by astroglia, a cell type that produces inhibitory CSPGs at sites of neural injury. Since CS chains are added post-translationally, using a relevant cell type could confirm binding with appropriately modified endogenous CSPGs. These experiments used mouse C8-D1A astrocytes, which express neurocan, display it on the cell surface, and deposit proteolytically processed neurocan fragments into the extracellular matrix. Chan et al., 55 Glia 369 (2007). PTPσ fusion proteins were found to bind astrocyte cultures, as shown by quantitative binding (p<0.001; FIG. 1F) and immunofluorescence (data not shown). PTPσ-Fc immunofluorescence showed binding over cell surfaces and extracellular matrix; Fc control gave no visible fluorescence. Pre-incubation with anti-CS antibody reduced binding. Also, PTPσ-Fc co-immunoprecipitated neurocan fragments from C8-D1A astrocyte astrocytes (data not shown). Involvement of CS chains was confirmed, by pre-treatment of astrocytes with chondroitinase ABC, or by pre-blocking with anti-CS antibody (p<0.01; FIGS. 1F and 1G). These treatments did not eliminate all PTPσ binding, suggesting either that the chondroitinase treatments partially digested the CS, or that PTPσ also binds to molecular epitopes other than CS, such as keratan sulfate chains. Confirmation of the role of CS in this interaction indicates that PTPσ binds not only to neurocan and aggrecan but also to other CSPGs produced by astrocytes.

### Example 2

### PTPσ Mediates the Inhibitory Effects of CSPG on Neuronal Regeneration

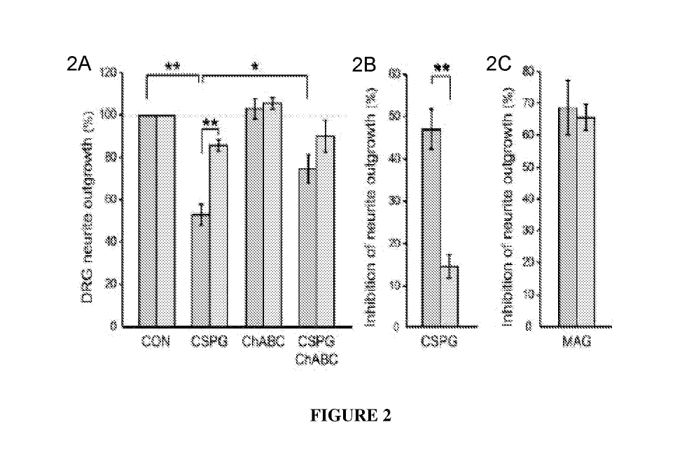

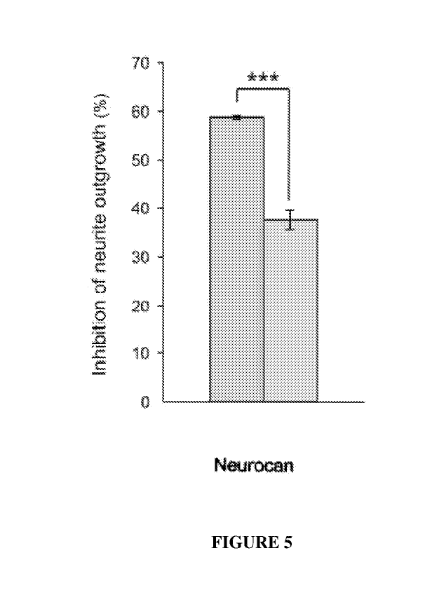

Having identified a binding interaction between PTPσ and CSPGs, studies were undertaken to test whether PTPσ is functionally involved in the inhibitory effects of CSPG on neurons. DRG neurons express high levels of PTPσ throughout life. Haworth et al., 12 Mol. Cell. Neurosci. 93 (1998). Postnatal day 8 (P8) dorsal root ganglion (DRG) neurons from mice with a targeted gene disruption of PTPσ (Elchebly et al., 21 Nat. Genet. 330 (1999)), or wild type controls, were cultured in the presence of a neural CSPG mixture (FIG. 2—DRG neurons from P8 mice were grown for 18 hours, then treated for 24 hours with or without CSPG, and visualized by GAP-43 immunolabeling.) or purified neurocan (FIG. 5 and data not shown). The CSPG mixture reduced control DRG neurite outgrowth by approximately 50% (FIGS. 2A and 2B) but had far less effect on neurons from PTPσ^{-/-} mice (p<0.01; 2A and 2B), showing a functional involvement of PTPσ in the response of young DRG neurons to inhibitory CSPGs.

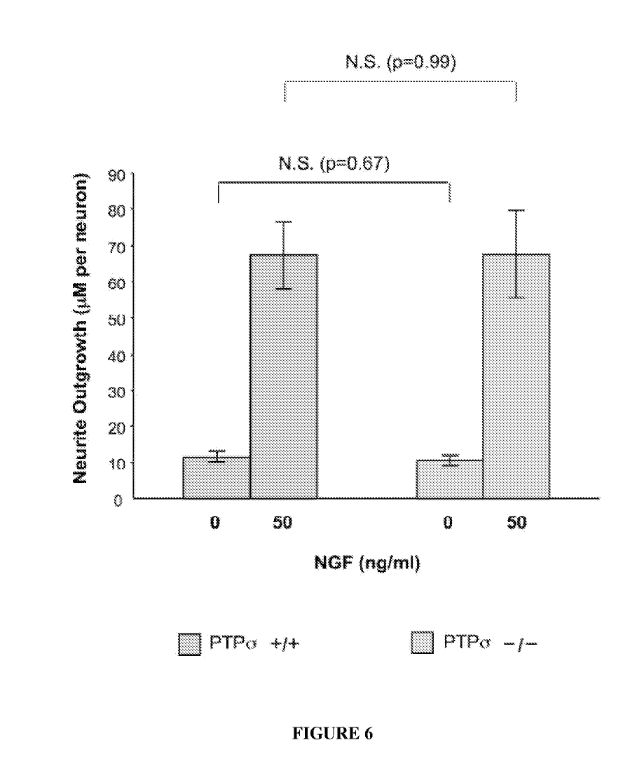

Comparable results were seen when neurons were challenged with purified neurocan (p<0.001; FIG. 6). DRG neurons from postnatal day 8-12 wild type or PTPσ^{-/-} mice were dissociated and plated on slides coated with or without neurocan (5 μg/ml). Cells were grown for 48 hours before immunostaining, and total length of GAP-43 positive neurites was quantified with Metamorph software. The observation of some remaining inhibitory effect of CSPGs on PTPσ^{-/-} neurons (FIG. 2B; FIG. 5) suggested the possible presence of additional receptors, which could be other PTPs in the LAR family, or receptors in other families, or there could be additional receptor-independent mechanisms.

To address the role of CS chains, the CSPG mixture was pretreated with chondroitinase ABC. This reduced its inhibitory effect on wild type DRG neurons (p<0.05), but did not cause a significant effect on PTPσ^{-/-} neurons; thus, the outgrowth difference between wild type and PTPσ^{-/-} neurons was no longer statistically significant (FIG. 2A). This is consistent with the results showing that CS chains are involved in binding to PTPσ (FIG. 1; FIG. 4).

Experiments were carried out to evaluate whether the effect of PTPσ deficiency shows specificity for CSPG. Using myelin-associated glycoprotein (MAG), a different inhibitory molecule (Domeniconi et al., 2005), as well as NGF, which can oppose the effect of myelin-associated inhibitors. Gao et al., 44 Neuron 609 (2004). PTPσ deficiency did not affect neurite outgrowth in response to either MAG (FIG. 2C; p=0.75) or NGF (FIG. 6; p=0.67 without NGF; p=0.99 with NGF). Thus, PTPσ shows a specific functional role in the inhibitory response of DRG neurons to CSPG.

### Example 3

### PTPσ as a Marker for Neuronal Injury

Tests were carried out to determine whether PTPσ has appropriate binding specificity to detect endogenous CSPG at sites of neural injury. In particular, experiments focused on whether PTPσ could preferentially recognize injured versus uninjured adult CNS tissue. The answer could not be deduced simply from its ability to bind CSPGs (FIG. 1), because PTPσ also binds HSPGs and potentially other ligands. Receptor ectodomain fusion proteins can be used to detect the distribution of their cognate ligands in tissues. Flanagan et al., 327 Meth. Enzymol. 19 (2000).

Immunofluorescence was performed on sections from the spinal cord lesion site and from unlesioned spinal cord. The sections were double fluorescence labeled with anti-neurocan antibody (Ncn), together with either PTPσ-Fc probe, or Fc control. In the unlesioned spinal cord, anti-neurocan labeled a thin line at the pia. PTPσ-Fc showed no labeling noticeably above Fc control. In the spinal cord, 7 days after dorsal hemisection, the lesion site showed simultaneous elevation of neurocan immunolabeling and PTPσ-Fc binding. The distributions overlapped, with PTPσ-Fc labeling additional areas. Chondroitinase ABC treatment reduced binding of PTPσ-Fc to the lesion site (adjacent sections were preincubated with or without chondroitinase ABC, then double labeled with anti-CS and PTPσ-Fc). Introduction of ΔLys mutation into a PTPσ-AP probe reduced lesion site binding close to AP tag control levels.

PTPσ-Fc did not show obvious binding above background on uninjured spinal cord (data not shown), whereas it showed strong binding around the lesion site a week after spinal cord injury (data not shown). PTPσ-Fc labeling overlapped with neurocan immunolabeling, which was also induced by injury as expected (data not shown), although the patterns were not identical, with PTPσ-Fc appearing to label additional areas, consistent with binding to additional CSPGs. Binding was reduced by chondroitinase ABC treatment, or by use of the PTPσ ΔLys mutant (data not shown), once again confirming involvement of the CS moiety. These binding results on spinal cord provide further evidence for the role of PTPσ as a CSPG receptor, and also show that it has appropriate binding specificity to serve biologically as a selective detector for injury sites in the adult CNS.

### Example 4

### PTP Mediates the Inhibitor Effects of CSPG on Neuronal Regeneration in Adult Animals

To test further for relevance to spinal cord injury, two functional models were used. After spinal cord injury, reactive glia produce an increasing gradient of CSPG beginning in the lesion penumbra and increasing toward the epicenter. In vivo, regenerating axons within this gradient stall and display dystrophic growth cone morphology. Davies 390 Nature 680 (1997). The dystrophic growth cone can be produced in vitro when neurons are exposed to a gradient of CSPG. Tom et al., 2004. A four-fold increase was detected in PTPσ^{-/-} neurites crossing the inhibitory outer rim of the gradient versus wild type controls (p<0.001; FIG. 3A). Adult wild type DRG neurons were visualized by β-tubulin antibody and seen to avoid the most inhibitory rim of the gradient visualized by anti-CS antibody (data not shown). PTPσ^{-/-} neurons showed greater ability to cross the rim (data not shown). Using an in vivo spinal cord injury model, a dorsal column crush injury was performed on adult mice, and the position of labeled sensory axons in the fasciculus gracilis was examined 14 days later. Confocal images were taken of longitudinal sections from adult mouse spinal cord 14 days after dorsal column crush, caudal to the left. Sensory axons were labeled with DexTR and the lesion was delineated by GFAP+ astrocytes. Wild type axons were seen several hundred microns from the lesion center; PTPσ^{-/-} axons abuted the edge of the lesion core (data not shown). In the PTPσ^{-/-} mice, axon extension into the lesion penumbra was significantly improved (p<0.002; FIG. 3B; FIG. 7). The extent of the axons was assessed by measuring to a “fiber front”, defined as the point at which there is a fasicle of contiguous fibers approximately 30 μm wide or more. Confocal z-stack images were taken of PTPσ^{-/-} mouse dorsal column crush lesion, showing relationship between injured fibers, anti-CS labeling, and reactive astrocyte. PTPσ^{-/-} axons extended well into the region of inhibitory proteoglycan surrounding the lesion (data not shown). Similar to the effect of chondroitinase (Bradbury et al., 2002), robust regeneration beyond the core of the lesion did not occur. This observation may reflect partial redundancy with other PTPs in the LAR subfamily, and it would also be consistent with the known presence of other growth impediments such as the myelin inhibitors (Case & Tessier-Lavigne, 2005; Domeniconi et al., 2005; Liu et al., 2006; Lu et al., 2008; Yiu & He, 2006), and factors intrinsic to unconditioned neurons (Goldberg et al., 296 Science 1860 (2002); Neumann & Woolf, 23 Neuron 83 (1999)). The results in this regeneration model system demonstrate a role for PTPσ in mediating the axonal response to the inhibitory CSPG-rich scar in a spinal cord lesion in vivo.

CSPG is one of the major inhibitors of neural regeneration; however, prior to the present invention, the mechanism has been poorly understood, and it has been unclear if this even involves specific cellular receptors, limiting the options to tackle this important area by molecular approaches. Finding that PTPσ is a functional receptor that binds and mediates actions of CSPGs indicates molecular approaches to manipulate CSPG action not only in regeneration, but also in development and plasticity. Other PTPs of the LAR subfamily may collaborate in nerve regeneration.

The finding that a PTPσ fusion protein can detect lesion sites in the adult CNS not only sheds light on its biological role, but also provides an injury biomarker, and thus a tool for research or diagnosis. Furthermore, identification of a specific site on PTPσ that binds CSPG represents an intervention for drugs to treat spinal cord injury and other CNS injuries and disorders. Blocking approaches include, for example using soluble receptor ectodomains. Such approaches are optionally combined with blockade of other regeneration inhibitors. These therapeutic approaches are relevant to many other forms of neural injury as well as neurodegeneration that involve reactive astrogliosis. These therapeutic interventions enhance regeneration or plasticity after nervous system injury.

1. A method of promoting outgrowth of a neuron, comprising contacting the neuron with an agent that inhibits the interaction of chondroitin sulfate proteoglycan (CSPG) to protein tyrosine phosphatase sigma (PTPσ).

2. The method of claim 1, wherein the neuron is in vivo.

3. The method of claim 1, wherein the neuron is in vitro.

4. A method of promoting neural regeneration in the central nervous system (CNS) of a subject in need thereof, comprising contacting a neuron of the subject with an agent that inhibits the interaction of chondroitin sulfate proteoglycan (CSPG) to protein tyrosine phosphatase sigma (PTPσ).

5. The method of claim 4, wherein the agent inhibits binding of CSPG to PTPσ.

6. The method of claim 5 wherein the neuron is located at a site of injured or diseased tissue.

7. The method of claim 6 5, wherein the neuron is located at a site of spinal cord injury.

8. The method of claim 5, wherein the agent binds to the first immunoglobulin-like domain of PTPσ.

9. The method of claim 5, wherein the agent comprises a soluble PTPσ polypeptide or portion or fragment thereof.

10. The method of claim 5, wherein the agent comprises a soluble PTPσ ectodomain or a portion or fragment thereof.

11. The method of claim 5, wherein the agent comprises a molecule that binds to the CSPG binding site of PTPσ, or that binds to the PTPσ binding site of CSPG.

12. The method of claim 5, wherein the agent is a PTPσ-specific antibody or portion or fragment thereof, the epitope binding specificity of which comprises the first immunoglobulin-like domain of PTPσ.

13. The method of claim 5, further comprising contacting the neuron with a second agent, wherein the second agent inhibits a myelin inhibitor of neural regeneration.

14.-16. (canceled)

17. A method of inhibiting the interaction of chondroitin sulfate proteoglycan (CSPG) to transmembrane Protein Tyrosine Phosphatase sigma (PTPσ) in a cell, comprising contacting the cell with an agent that inhibits CSPG binding to PTPσ.

18. The method of claim 17, wherein the cell is a neuron.

19.-20. (canceled)

21. The method of claim 17, wherein the cell is a non-neuronal cell that provides support to neural cells.

22. The method of claim 18, wherein the agent is isolated/purified CSPG or a portion or fragment thereof.

23. The method of claim 22, wherein the fragment of CSPG is chondroitin sulfate or a portion thereof.

24. The method of claim 18, wherein the agent is isolated/purified antibody or fragment thereof which specifically binds an epitope on chondroitin sulfate, or an isolated/purified antibody or fragment thereof which specifically binds an epitope on the first immunoglobulin-like domain of PTPσ.

25.-27. (canceled)

28. The method of claim 18, wherein the agent is an isolated/purified soluble PTPσ polypeptide, or a purified/isolated soluble PTPσ ectodomain, or a small molecule.

29.-30. (canceled)


SEQUENCE LISTING (8 sequences)

SEQ ID NO: 1 - protein, 1505 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSISPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 2 - DNA, 6018 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggagccctgcagattgaaagcagtgaggaaaccgaccagggcaaatatgagtgtgtggccaccaacagcgccggcgtgcgctactcctcacctgccaacctctacgtgcgagagcttcgagaagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccatctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccaggcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 3 - protein, 1501 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSISPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 4 - DNA, 6006 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcaggagccctgcagattgaaagcagtgaggaaaccgaccagggcaaatatgagtgtgtggccaccaacagcgccggcgtgcgctactcctcacctgccaacctctacgtgcgagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccatctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccaggcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 5 - protein, 1948 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 6 - DNA, 7347 bp, Homo sapiens
cctcgcgccgcccgcccggcagcccggccggcgcgcgcacgccgcgagccgctggcgctcgggctccgctcggatcccatgcaacagccacgatgtgaagcggggcagagccgggggagcccagcccagccagcctccagacgttgccccatctgacgctcggctcgaggcctctctgtgagggaccggggggccatccccctccagggcggagatcggaggtcgctgccaagcatggcgcccacctggggccctggcatggtgtctgtggttggtcccatgggcctccttgtggtcctgctcgttggaggctgtgcagcagaagagccccccaggtttatcaaagaacccaaggaccagatcggcgtgtcggggggtgtggcctctttcgtgtgtcaggccacgggtgaccccaagccacgagtgacctggaacaagaagggcaagaaggtcaactctcagcgctttgagacgattgagtttgatgagagtgcaggggcagtgctgaggatccagccgctgaggacaccgcgggatgaaaacgtgtacgagtgtgtggcccagaactcggttggggagatcacagtccatgccaagcttactgtcctccgagaggaccagctgccctctggcttccccaacatcgacatgggcccacagttgaaggtggtggagcggacacggacagccaccatgctctgtgcagccagcggcaaccctgaccctgagatcacctggttcaaggacttcctgcctgtggatcctagtgccagcaatggacgcatcaaacagctgcgatcagaaacctttgaaagcactccgattcgaggagccctgcagattgaaagcagtgaggaaaccgaccagggcaaatatgagtgtgtggccaccaacagcgccggcgtgcgctactcctcacctgccaacctctacgtgcgagagcttcgagaagtccgccgcgtggccccgcgcttctccatcctgcccatgagccacgagatcatgccagggggcaacgtgaacatcacctgcgtggccgtgggctcgcccatgccatacgtgaagtggatgcagggggccgaggacctgacccccgaggatgacatgcccgtgggtcggaacgtgctggaactcacagatgtcaaggactcggccaactacacctgcgtggccatgtccagcctgggcgtcattgaggcggttgctcagatcacggtgaaatctctccccaaagctcccgggactcccatggtgactgagaacacagccaccagcatcaccatcacgtgggactcgggcaacccagatcctgtgtcctattacgtcatcgaatataaatccaagagccaagacgggccgtatcagattaaagaggacatcaccaccacacgttacagcatcggcggcctgagccccaactcggagtacgagatctgggtgtcggccgtcaactccatcggccaggggccccccagcgagtccgtggtcacccgcacaggcgagcaggccccggccagcgcgccgcggaacgtgcaagcccggatgctcagcgcgaccaccatgattgtgcagtgggaggagccggtggagcccaacggcctgatccgcggctaccgcgtctactacaccatggaaccggagcaccccgtgggcaactggcagaagcacaacgtggacgacagcctgctgaccaccgtgggcagcctgctggaggacgagacctacaccgtgcgggtgctcgccttcacctccgtcggcgacgggcccctctcggaccccatccaggtcaagacgcagcagggagtgccgggccagcccatgaacctgcgggccgaggccaggtcggagaccagcatcacgctgtcctggagccccccgcggcaggagagtatcatcaagtacgagctcctcttccgggaaggcgaccatggccgggaggtgggaaggaccttcgacccgacgacttcctacgtggtggaggacctgaagcccaacacggagtacgccttccgcctggcggcccgctcgccgcagggcctgggcgccttcacccccgtggtgcggcagcgcacgctgcagtccaaaccgtcagccccccctcaagacgttaaatgtgtcagcgtgcgctccacggccattttggtaagttggcgcccgccgccgccggaaacgcacaacggggccctggtgggctacagcgtccgctaccgaccgctgggctcagaggacccggaacccaaggaggtgaacggcatccccccgaccaccactcagatcctgctggaggccttggagaagtggacccagtaccgcatcacgactgtcgctcacacagaggtgggaccagggcccgagagctcgcccgtggtcgtccgcaccgacgaggatgtgcccagcgcgccgccgcggaaggtggaggcggaggcgctcaacgccacggccatccgcgtgctgtggcgctcgcccgcgcccggccggcagcacggccagatccgcggctaccaggtccactacgtgcgcatggagggcgccgaggcccgcgggccgccgcgcatcaaggacgtcatgctggccgatgcccagtgggagacggatgacacggccgaatatgagatggtcatcacaaacttgcagcctgagaccgcgtactccatcacggtagccgcctacaccatgaagggcgatggcgctcgcagcaaacccaaggtggttgtgaccaagggagcagtgctgggccgcccaaccctgtcggtgcagcagacccccgagggcagcctgctggcacgctgggagcccccggctggcaccgcggaggaccaggtgctgggctaccgcctgcagtttggccgtgaggactcgacgcccctggccaccctggagttcccgccctccgaggaccgctacacggcatcaggcgtgcacaagggggccacgtatgtgttccggcttgcggcccggagccgcggcggcctgggcgaggaggcagccgaggtcctgagcatcccggaggacacgccccgtggccacccgcagattctggaggcggccggcaacgcctcggccgggaccgtccttctccgctggctgccacccgtgcccgccgagcgcaacggggccatcgtcaaatacacggtggccgtgcgggaggccggtgccctgggccctgcccgagagactgagctgccggcagcggctgagccgggcgcggagaacgcgctcacgctgcagggcctgaagcccgacacggcctatgacctccaagtgcgagcccacacgcgccggggccctggccccttcagcccccccgtccgctaccggacgttcctgcgggaccaagtctcgcccaagaacttcaaggtgaaaatgatcatgaagacatcagttctgctcagctgggagttccctgacaactacaactcacccacaccctacaagatccagtacaatgggctcacactggatgtggatggccgtaccaccaagaagctcatcacgcacctcaagccccacaccttctacaactttgtgctgaccaatcgcggcagcagcctgggcggcctccagcagacggtcaccgcctggactgccttcaacctgctcaacggcaagcccagcgtcgcccccaagcctgatgctgacggcttcatcatggtgtatcttcctgacggccagagccccgtgcctgtccagagctatttcattgtgatggtgccactgcgcaagtctcgtggaggccaattcctgaccccgctgggtagcccagaggacatggatctggaagagctcatccaggacatctcacggctacagaggcgcagcctgcggcactcgcgtcagctggaggtgccccggccctatattgcagctcgcttctctgtgctgccacccacgttccatcccggcgaccagaagcagtatggcggcttcgataaccggggcctggagcccggccaccgctatgtcctcttcgtgcttgccgtgcttcagaagagcgagcctacctttgcagccagtcccttctcagaccccttccagctggataacccggacccccagcccatcgtggatggcgaggaggggcttatctgggtgatcgggcctgtgctggccgtggtcttcataatctgcattgtcattgctatcctgctctacaagaacaaacccgacagtaaacgcaaggactcagaaccccgcaccaaatgcctcctgaacaatgccgacctcgcccctcaccaccccaaggaccctgtggaaatgagacgcattaacttccagactccagattcaggcctcaggagccccctcagggagccggggtttcactttgaaagcatgcttagccacccgccaattcccatcgcagacatggcggagcacacggagcggctcaaggccaacgacagcctcaagctctcccaggagtatgagtccatcgaccctggacagcagttcacatgggaacattccaacctggaagtgaacaagccgaagaaccgctatgccaacgtcatcgcctatgaccactcccgtgtcatcctccagcccattgaaggcatcatgggcagtgattacatcaatgccaactacgtggacggctaccggtgtcagaacgcgtacattgccacgcaggggccgctgcctgagacctttggggacttctggcgtatggtgtgggagcagcggtcggcgaccatcgtcatgatgacgcggctggaggagaagtcacggatcaagtgtgatcagtattggcccaacagaggcacggagacctacggcttcatccaggtcacgttgctagataccatcgagctggccacattctgcgtcaggacattctctctgcacaagaatggctccagtgagaaacgcgaggtccgccagttccagtttacggcgtggccggaccatggcgtgcccgaatacccaacgcccttcctggctttcctgcggagagtcaagacctgcaacccgccagatgccggccccatcgtggttcactgcagtgccggtgtgggccgcacaggctgctttatcgtcatcgacgccatgcttgagcggatcaagccagagaagacagtcgatgtctatggccacgtgacgctcatgaggtcccagcgcaactacatggtgcagacggaggaccagtacagcttcatccacgaggccctgctggaggccgtgggctgtggcaacacagaagtgcccgcacgcagcctctatgcctacatccagaagctggcccaggtggagcctggcgaacacgtcactggcatggaactcgagttcaagcggctggctaactccaaggcccacacgtcacgcttcatcagtgccaatctgccttgtaacaagttcaagaaccgcctggtgaacatcatgccctatgagagcacacgggtctgtctgcaacccatccggggtgtggagggctctgactacatcaacgccagcttcattgatggctacaggcagcagaaggcctacatcgcgacacaggggccgctggcggagaccacggaagacttctggcgcatgctgtgggagaacaattcgacgatcgtggtgatgctgaccaagctgcgggagatgggccgggagaagtgtcaccagtactggccggccgagcgctctgcccgctaccagtactttgtggtagatccgatggcagaatacaacatgcctcagtatatcctgcgagagttcaaggtcacagatgcccgggatggccagtcccggactgtccggcagttccagttcacagactggccggaacagggtgtgccaaagtcgggggagggcttcatcgacttcattggccaagtgcataagactaaggagcagtttggccaggacggccccatctctgtccactgcagtgccggcgtgggcaggacgggcgtcttcatcacgcttagcatcgtgctggagcggatgcggtatgaaggcgtggtggacatctttcagacggtgaagatgctacgaacccagcggccggccatggtgcagacagaggatgagtaccagttctgttaccaggcggcactggagtacctcggaagctttgaccactatgcaacctaaagccatggttccccccaggcccgacaccactggccccggatgcctctgcccctcccgggcggacctcctgaggcctggacccccagtgggcagggcaggaggtggcagcggcagcagctgtgtttctgcaccatttccgaggacgacgcagcccctcgagcccccccaccggccccggccgccccagcgacctccctggcaccggccgccgccttcaaatacttggcacattcctcctttccttccaattccaaaaccagattccggggtggggggtggggggatggtgagcaaataggagtgctccccagaaccagaggagggtggggcacagaccatagacggacccctcgtcctcccccagcggtggtagggggacccggggggctcctccccgctctgcagcctggggacactgggctgggaccagaatccagctttcttttaaaactctcagtgtaactgtatcccgtgacatttcattttttttaaatagtgtatttttttttccatttttttttttaagagaaacaaacaaaagactcgccagtcaatgactttcaaagagaactaactttggcttattcatattctgttcaaagacagtctattttttcactgtagaaagcgtccttgtgtgatagttacgttcgcaaacgcgcacgccaggcccatggctgtaccttggcttttttttttttttttttttttttaatttttcctaccatcagaaagtgtgctttgctcacagaagaatgggatgtccttttttctttcttggctttttttttccccctttttgtttcatttttataaattaaattttcagacatatcaaatacagttctgagggtaaggtcatgggggagctcggacccagtggcgttgggtgcggttgagggggacgctgctgtaagaggagagagatgacagtggtcctcctctgagagcctgagctgtctccccgtctcccgcccccaaggagacagagaggatcctacttcttcggggacagtggctgtatggctgtgctgccccacatcagggaccctttccccctgggactgtggggcagtttgggagcaaaaccagaaggacaggcccccctctacccgcctaccctgagcaagcgagttgttcctctttgtacaagggcaggtctgcggttactttcaacactgtttattccagcggaagcagccgggtggttttcccacccccgtgtatgtagatatatcgactttgtattaaaggaagatcgtctga

SEQ ID NO: 7 - protein, 1907 aa, Mus musculus
MAPTWSPSVVSVVGPVGLFLVLLARGCLAEEPPRFIREPKDQIGVSGGVASFVCQATGDPKPRVTWNKKGKKVNSQRFETIDFDESSGAVLRIQPLRTPRDENVYECVAQNSVGEITIHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPVVTENTATSITVTWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEAKSETSIGLSWSAPRQESVIKYELLFREGDRGREVGRTFDPTTAFVVEDLKPNTEYAFRLAARSPQGLGAFTAVVRQRTLQAKPSAPPQDVKCTSLRSTAILVSWRPPPPETHNGALVGYSVRYRPLGSEDPDPKEVNNIPPTTTQILLEALEKWTEYRVTAVAYTEVGPGPESSPVVVRTDEDVPSAPPRKVEAEALNATAIRVLWRSPTPGRQHGQIRGYQVHYVRMEGAEARGPPRIKDIMLADAQEMVITNLQPETAYSITVAAYTMKGDGARSKPKVVVTKGAVLGRPTLSVQQTPEGSLLARWEPPADAAEDPVLGYRLQFGREDAAPATLELAAWERRFAAPAHKGATYVFRLAARGRAGLGEEAAAALSIPEDAPRGFPQILGAAGNVSAGSVLLRWLPPVPAERNGAIIKYTVSVREAGAPGPATETELAAAAQPGAETALTLRGLRPETAYELRVRAHTRRGPGPFSPPLRYRLARDPVSPKNFKVKMIMKTSVLLSWEFPDNYNSPTPYKIQYNGLTLDVDGRTTKKLITHLKPHTFYNFVLTNRGSSLGGLQQTVTARTAFNMLSGKPSVAPKPDNDGFIVVYLPDGQSPVTVQNYFIVMVPLRKSRGGQFPVLLGSPEDMDLEELIQDISRLQRRSLRHSRQLEVPRPYIAARFSILPAVFHPGNQKQYGGFDNRGLEPGHRYVLFVLAVLQKNEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPITDMAEHMERLKANDSLKLSQEYESIDPGQQFTWEHSNLEANKPKNRYANVIAYDHSRVILQPLEGIMGSDYINANYVDGYRRQNAYIATQGPLPETFGDFWRMVWEQRSATVVMMTRLEEKSRIKCDQYWPNRGTETYGFIQVTLLDTMELATFCVRTFSLHKNGSSEKREVRHFQFTAWPDHGVPEYPTPFLAFLRRVKTCNPPDAGPIVVHCSAGVGRTGCFIVIDAMLERIKTEKTVDVYGHVTLMRSQRNYMVQTEDQYGFIHEALLEAVGCGNTEVPARSLYTYIQKLAQVEPGEHVTGMELEFKRLASSKAHTSRFITASLPCNKFKNRLVNILPYESSRVCLQPIRGVEGSDYINASFIDGYRQQKAYIATQGPLAETTEDFWRALWENNSTIVVMLTKLREMGREKCHQYWPAERSARYQYFVVDPMAEYNMPQYILREFKVTDARDGQSRTVRQFQFTDWPEQGAPKSGEGFIDFIGQVHKTKEQFGQDGPISVHCSAGVGRTGVFITLSIVLERMRYEGVVDIFQTVKVLRTQRPAMVQTEDEYQFCFQAALEYLGSFDHYAT

SEQ ID NO: 8 - DNA, 6874 bp, Mus musculus
aggggtgacgtcaccggctgggggcgcgcgagccgcagtggggttttgccccgcccgccaggcagctcgggccgcgcgcacacgcggagccgccggagcccgggccgacccggtgccgggagcagcatgcggagcccgcagacgctgcccctctggacacctcagcctgaggcctctccgtgagtcacgggggtaccatcccccaccagggcagaggctggaggccactgccaagcatggcgcccacctggagtcccagcgtggtgtctgtggtgggtcctgtggggctcttcctcgtactgctggccagaggatgcttggctgaagaaccacccaggtttatcagagagcccaaggatcagattggagtgtcgggaggcgtggcctccttcgtgtgccaggccacgggtgatcctaagccacgggtgacctggaacaagaagggcaagaaagtgaactcacagcgcttcgagaccattgactttgacgagagctctggggcggtcctgaggatccagccacttcggacgcctcgggatgagaacgtgtacgagtgtgtggcccagaactcggtgggcgaaatcacaattcatgcaaagctcaccgtccttcgagaggaccagctgcctcctggcttccccaacattgacatgggcccccagttgaaggttgtagagcgcacacgcacagccaccatgctctgtgctgccagcgggaacccggaccctgagatcacctggtttaaggacttcctgcctgtggaccccagtgccagcaacgggcggatcaagcagcttcgatcaggtgccctgcagattgagagcagcgaggagacagaccagggcaagtacgagtgtgtggccaccaacagcgctggggtgcgctactcatcacctgccaacctctacgtgcgagtccgccgtgtggccccacgcttctccatcctgcccatgagccacgagatcatgcccggtgggaatgtgaatatcacttgtgtggccgtgggctcacccatgccctacgtgaaatggatgcagggggccgaggacctgacgcctgaggatgacatgcccgtgggtcggaatgttctagaactcacggatgtcaaggactcagctaactacacttgtgtggccatgtccagcctgggtgtgatcgaggccgtggcccagatcactgtaaaatctctccccaaagcccctgggactcctgtggtgacggagaacactgccaccagtatcactgtcacatgggactcgggcaaccctgaccccgtgtcctactacgtaattgagtataagtccaaaagccaggatgggccgtatcagatcaaagaagacatcaccaccacgcgctacagcatcggaggcctgagccccaattctgagtatgagatctgggtgtcagctgtcaactccattggccagggccctcccagtgaatcggtggtgacccgcacaggtgagcaggcaccagccagcgctcccaggaatgttcaggcccgcatgctcagcgccaccaccatgatcgtgcagtgggaggagcctgtggagcccaatggcctgatccgtggctaccgtgtctactataccatggagccggaacacccagtgggcaactggcagaaacacaatgtggacgacagtctcctgaccactgtgggcagcctgctggaagacgagacctacaccgtgcgcgtgctcgccttcacgtcggtgggcgacggaccactgtcagaccccatccaggtcaagacccagcagggagttcctggccagcccatgaacttgcgggctgaggccaagtcagagaccagcattgggctctcgtggagtgcaccacgacaggagagtgtcattaagtatgaactgctcttccgggagggcgaccgaggccgagaggtggggcgaaccttcgacccaaccacagcctttgtggtggaggacctcaagcccaatacggagtatgcgttccggctggcggcgcgctcgccgcagggcctgggcgccttcaccgcggtcgtgcgccagcgcacgctgcaggccaaaccgtcagccccccctcaagacgttaagtgcaccagcttgcgctccacggccatattggtaagttggcgcccgccaccgccagaaactcacaacggggccctcgtgggctacagcgtccgctaccgaccgctgggctcagaggacccggaccccaaggaggtgaacaacatacccccgaccaccactcagatccttctggaagctttggagaaatggacggagtaccgtgtcaccgccgtggcttacacagaggtgggaccagggcccgagagctcgcccgtggtcgtccgcaccgatgaggacgtgcccagcgcgcccccgcggaaggtggaggcggaggcgctcaacgccacagccatccgagtgctgtggcgctcgcccacgcccggccggcagcacgggcagatccgcggctaccaggtccactatgtgcgcatggagggtgccgaggcccgcgggccaccgcgcatcaaggacatcatgctggcggatgcccaggaaatggtgataacgaacctccagcctgagactgcttactctatcacagtagccgcgtataccatgaaaggcgatggcgctcgcagcaaaccgaaggtggtggtgaccaagggagcagtgctgggccgccccaccctgtcggtgcagcagacccccgagggcagcctgctggcgcgctgggagccccccgcggacgcggccgaggacccggtgcttggctaccgcctgcagtttgggcgcgaagacgcggccccggccacgttggagctggctgcgtgggagcggcggttcgcggcgcctgcacacaagggcgccacctatgtgttccggctggcagcgcggggccgcgcggggttgggcgaggaggccgcggcagcgctgagcatccccgaggacgctccgcgcggcttcccgcagatcttgggcgccgcgggcaacgtgtccgcgggctccgtgctactgcgctggctgccacccgtgcccgccgagcgcaacggcgccatcatcaagtacacggtgtccgtgcgggaggccggcgcccctgggcccgcgaccgagacggagctggcggcggccgcccagccgggggccgagacagcgctcacgctgcgagggctgcggccggagacggcctacgagttacgcgtgcgcgcacacacgcgtcgcggcccgggccccttctcacccccgctgcgctacaggctcgcgcgggacccagtctccccaaagaacttcaaggtgaagatgatcatgaagacttcagtgctgctgagctgggagttccccgacaactataactcacccacaccctacaagattcagtacaatgggctcaccctggatgtggacggccgcacgaccaagaagctgatcacacacctcaagccacacaccttctataatttcgtgctcaccaaccgtggcagcagcctggggggcctgcagcagacggtcactgccaggaccgcctttaacatgctcagtggcaagcctagcgtcgccccgaagcccgacaatgacggtttcatcgtggtctacctgcctgatggccagagtcctgtgaccgtgcagaactacttcattgtgatggtcccacttcggaagtctcgaggtggccagttccctgtcctactaggtagtccagaggacatggatctggaggagctcatccaggacatctcccggctgcagaggcgcagcctgcgccactccagacagctggaggtgcctcggccctacatcgccgctcgattctccatcctgccagctgtcttccatcctgggaaccagaagcaatatggtggctttgacaacaggggcttggagccaggccaccgctatgtcctctttgtgcttgctgtgttgcagaagaatgagcctacatttgcagccagtcccttctcagaccccttccagctggacaacccggaccctcagcccattgtggacggcgaggagggcctcatctgggtgattgggcctgtgctggccgtggtcttcatcatctgcatcgtgattgccatcctgctgtacaagaacaaacctgacagcaaacgcaaggactcagagccccgcaccaaatgcttactgaacaatgccgaccttgccccccatcaccccaaggaccctgtggaaatgcgacgcatcaacttccagacaccaggtatgctcagccacccacccatccccatcacagacatggcggagcacatggagagactcaaagccaacgacagcctgaagctctcccaggagtacgagtccattgaccccgggcagcaattcacgtgggaacattcgaacctggaggccaacaagcccaagaaccgctatgccaacgtcatcgcctatgaccactcacgagtcatcctgcagcccctagaaggcatcatgggtagtgattacatcaatgccaactatgtggacggctaccggcggcagaatgcatacattgccacgcaggggcccctgcctgagacctttggggacttctggcggatggtgtgggagcagcgatcggccactgtggtcatgatgacgcgactggaggagaaatcacggatcaaatgtgaccaatactggcctaaccgaggcaccgagacatacggcttcatccaggtcaccctactagataccatggagctggctaccttctgcgtcaggactttttctctacacaagaatggctctagcgagaagcgtgaggtgcgacatttccagttcacggcatggcccgaccacggggtacctgagtaccccacgcccttcctggcattcctgcgaagagtcaagacctgcaacccgcctgatgctggccccattgtggtccactgcagcgcgggtgtggggcgcactggctgcttcatcgtaattgacgccatgctagagcgcatcaagacagagaagaccgtggatgtgtatggacatgtgacactcatgcggtcgcagcgcaactacatggtgcagacagaggatcagtatggcttcatccacgaggcgctgctggaggctgtgggctgcggcaataccgaggtccctgctcgcagcctctacacctacatccagaagctggcccaggtggagcctggcgagcacgtcacgggcatggagcttgagttcaagaggctcgccagttccaaggcacacacttcgcgcttcatcaccgccagcctgccttgcaacaagtttaagaaccgactggtgaacatcctgccgtacgagagctcgcgtgtctgcctgcagcccatccgcggtgtggagggctctgactacatcaatgccagctttatcgacggctatagacagcagaaagcctacattgcaacacaggggccactggcagagaccacagaggacttctggcgagctctgtgggagaacaactctactattgtcgtaatgctcaccaagctccgagaaatgggccgggaaaagtgccaccagtactggccagccgagcgctctgcccgctaccagtactttgtggttgacccgatggcagagtataacatgccacagtacattctgcgtgagtttaaggtcacagatgcccgggatggccagtcccggaccgtccgacagttccagttcacggactggccagagcagggtgcacccaagtcaggggaaggcttcattgacttcatcggccaagtgcataagaccaaggagcagtttggccaggacggacccatctcagtgcactgcagcgccggagtgggcaggaccggagtgttcatcaccctgagcatcgtgcttgagcggatgcgctacgagggcgtggtggacattttccagacagtgaaggtgcttcggacccagaggcctgccatggtgcagacagaggacgagtaccagttctgcttccaggcggctttggaatacctgggcagttttgatcattatgcaacataagccatgggccccgcccaacacctcagccctgcgccaagtgccctggatgtgagcctaggcccgccgctgggcaggatgcggcccagggagacctcctcttcgcggagacaggcgctgccttcctcattcccttctgattccaaaacgaggttccagggtggggggttggggtggagagagaaggagccactgctccccaggctggggtcacacagggaccgacctctgcttccgcactcccctgcctgccttttggcaacattttttttcttatttttttttaatagtgtatattttttttctttttctttttttcttttttttttttaagaaaaaaacaaaatcgtgccggtcaaaactttgaaaaagaaacaagatcactgtttgtgcctctgtgggaggcctattttttcatagttagtgtgccgtgtggcggctatgtgcggccacttcgacggcttctgtgtgtgcatctttcccacatgcccgacactgcccccatccccatgtgaatggtgcgcttagtttttatttttaacctttttactttttttttaatcaatcttcagacatatcagatatggagggtgaggcgctgggggcactcgggccagactacagggacatggccaccaaggacacagtggctggccttgctgctccagtccctggcacaccagggagggtcctcgtctactcatgacctctgtgccccgcatggaggacctgggactacgggacacttgggggatatccaaccccctggagcaactgaggtctctctttgtaggagagtgggtcagtactcgtccccgctgttttttgggcagaagcagcaggtgacgcccctgtatgtagataaaccaactttgtattaaagaaagattcgtccgacctagaaaaaaaaaaaaaaa